(19) United States
(12) Patent Application Publication
(10) Pub. No.: US 2015/0104519 A1
(43) Pub. Date: Apr. 16, 2015

(54) Pharmaceutical Compositions

(71) Applicant: Alpharma Pharmaceuticals LLC, Bridgewater, NJ (US)

(72) Inventors: Alfred Liang, Rahway, NJ (US); Frank Matthews, Bridgewater, NJ (US); Garth Boehm, Bridgewater, NJ (US); Lijuan Tang, Bridgewater, NJ (US); Frank Johnson, Bridgewater, NJ (US); Joseph Stauffer, Skillman, NJ (US)

(21) Appl. No.: 14/578,574

(22) Filed: Dec. 22, 2014

Related U.S. Application Data
(63) Continuation of application No. 12/808,204, filed on Jun. 15, 2010.
(60) Provisional application No. 61/007,940, filed on Dec. 17, 2007.

(51) Int. Cl. A61K 9/16; A61K 31/485

(52) U.S. Cl. CPC A61K 9/16; A61K 31/485
USPC 424/489

(57) ABSTRACT

Provided herein are formulations and methods for treating pain in human beings. Also provided are optimal ratios at which an opioid and an opioid antagonist may be combined for administration to humans such that the opioid activity is inhibited. These ratios may also be used to formulate compositions containing both an opioid and an opioid antagonist within a single pharmaceutical dosing unit.

### RELATED APPLICATIONS

This application claims priority to U.S. Ser. No. 61/007,940 filed Dec. 17, 2007.

### FIELD OF THE INVENTION

This invention pertains to compositions and methods useful for treating pain in human patients. One such composition contains both an opioid antagonist and an opioid agonist formulated such that the agonist is released over time with minimal release of the antagonist.

### BACKGROUND OF THE INVENTION

Improved methods for treating pain are desired by those of skill in the art. A disease in which pain is a major symptom is osteoarthritis (OA). OA is the most common form of arthritis in the United States (Hochberg et al., 1995a), affecting more than 21 million people. It is a disease of primarily middle-aged and older adults and is a leading cause of disability (American College of Rheumatology, 2000a). OA results from degeneration of the joint cartilage, and usually involves the neck, low back, knees, hips, and fingers. The prevalence of OA of the hip and knee increases progressively with age (Peloso et al., 2000). Unlike rheumatoid arthritis and other inflammatory arthritides, inflammation, if present, is usually mild and localized to the joint. The cause of OA is unknown, but biomechanical stresses affecting the articular cartilage and subchondral bone, biochemical changes in the articular cartilage and synovial membrane, and genetic factors are significant in its pathogenesis (Hochberg et al., 1995b; American College of Rheumatology, 2000b).

OA is characterized by pain that typically worsens with activity and weight bearing and improves with rest, as well as morning stiffness, and pain and stiffness that ease after a few minutes of movement. Clinical examination often reveals tenderness to palpation, bony enlargement, crepitus, and/or limited joint motion (American College of Rheumatology, 2000b). As the disease advances, OA patients experience increasing pain and loss of function, with pain intruding at periods of rest (Peloso et al., 2000). Since no cure for OA is available, the primary goal of OA treatment is to reduce pain while maintaining or improving joint mobility and limiting functional impairment.

Nonpharmacologic and pharmacologic treatments for OA are used in conjunction to reduce pain and to improve functional status. Nonpharmacologic therapies include patient education, weight loss (if overweight), occupational therapy, physical therapy, and aerobic exercise programs to restore joint movement and increase strength and aerobic capacity (American College of Rheumatology, 2000a). The initial pharmacologic therapies for OA include nonopioid analgesics (e.g., acetaminophen) and topical analgesics, followed by treatment with nonsteroidal anti-inflammatory drugs (NSAIDs) and judicious use of intra-articular steroid injections (Hochberg et al., 1995a). Although these medications may provide temporary pain relief′, the beneficial effect may be offset by other factors. Use of nonopioid analgesics to treat moderate to severe OA pain is limited by a ceiling effect for analgesia (Roth et al., 2000). Additionally, NSAIDs can be toxic to the gastrointestinal tract, and NSAIDs and acetaminophen can produce renal toxicity, especially in the elderly (Peloso et al., 2000). Thus, a need exists for additional analgesic treatment options for pain associated with OA.

Recent efforts have been made to liberalize the use of opioids for the treatment of chronic nonmalignant pain (Sullivan et al., 2005). Sullivan proposes subject-centered principles to guide efforts to relieve chronic nonmalignant pain, including the acceptance of all subject pain reports as valid but negotiation of treatment goals early in care, avoidance of subject harm, and incorporation of chronic opioids as one part of the treatment plan if they improve the subject's overall health-related quality of life. Prescribing opiates in the treatment of chronic nonmalignant pain may pose a challenge to the primary care physician (Olsen et al., 2004).

Although an outright ban on opioid use in chronic nonmalignant pain is no longer ethically acceptable, ensuring that opioids provide overall benefit to subjects requires significant physician time and skill. Subjects with chronic nonmalignant pain should be assessed and treated for concurrent psychiatric disorders; those with disorders are entitled to equivalent efforts at pain relief. The essential question is not whether chronic nonmalignant pain is real or proportional to objective disease severity, but how it should be managed so that the subject's overall quality of life is optimized.

As early as the mid 1990s, naltrexone has been shown to effectively block morphine effects in humans (Kaiko et al., 1995). Morphine effects in normal volunteers were blocked by three 100-mg doses of naltrexone. The first dose of naltrexone was given 24 hours before dosing with controlled release morphine sulfate (MS Contin®), followed by a second dose at the time of MS Contin dosing and a third dose 24 hours after MS Contin administration. Single 200 mg doses of MS Contin given with the naltrexone blockade were generally well tolerated, and adverse effects were similar to those reported for naltrexone alone and for lower doses of morphine without naltrexone. Naltrexone proved safe and effective in blocking the effects of controlled release morphine, permitting bioequivalence studies of a high dose of morphine in normal volunteers.

Although well absorbed orally, naltrexone is subject to significant first-pass metabolism, with oral bioavailability estimates ranging from 5% to 40% (Naltrexone HCl Tablets, USP Package Insert). The activity of naltrexone is believed to be due to both the parent compound and the 6-β-naltrexol metabolite. Both parent drug and metabolites are excreted primarily by the kidney (53% to 79% of the dose); however, urinary excretion of unchanged naltrexone accounts for less than 2% of an oral dose and fecal excretion is a minor elimination pathway. The mean elimination terminal half-life (t_{1/2}) values for naltrexone and 6-β-naltrexol are 4 hours and 13 hours, respectively. Naltrexone and 6-β-naltrexol are dose-proportional in terms of area under the concentration-time curve (AUC) and maximum plasma concentration (Cₘₐₓ) over the range of 50 to 200 mg and do not accumulate after 100 mg daily doses.

Various formulations of opioids are in development that have a reduced risk of diversion and non-medical use and can be used to treat patients with chronic, nonmalignant conditions. Kadian® (morphine sulfate extended-release capsule) was developed for use in subjects with chronic pain who require repeated dosing with a potent opioid analgesic, and has been tested in subjects with pain due to malignant and nonmalignant conditions. Kadian contains polymer-coated extended-release pellets of morphine sulfate, to deliver up to 24 hours of continuous pain relief. This formulation lacks an immediate-release component, only providing a slow release of the analgesic. This slow-release technology serves to minimize plasma peaks and troughs, thereby providing a relatively flat pharmacokinetic (PK) curve upon multiple dosing. This delivery mechanism is ideally suited for chronic pain patients. Kadian capsules are an extended-release oral formulation of morphine sulfate indicated for the management of moderate to severe pain when a continuous, around-the-clock opioid analgesic is needed for an extended period of time.

However, persons abusing opioids are likely to tamper with controlled-release formulations in hopes of obtaining the entire dose to induce an immediate euphoria. To further deter non-medical opioid use, formulations containing opioid antagonists are being developed. As described herein, Kadian NT (morphine sulfate plus naltrexone hydrochloride extended-release capsules), is a product that is intended to be used as an opiate analgesic for moderate to severe pain. Its abuse-deterrence feature incorporates an immediate release of naltrexone upon illicit manipulation; this is intended to neutralize the euphoric potential of morphine and increase safety after ingestion of the tampered product. If Kadian NT is used as directed, a patient should receive a dose of morphine equivalent to the same mg dose of Kadian. However, if the drug product is tampered with and ingested by a patient who is opioid dependent, the patient may be exposed to a dose of naltrexone sufficient to produce withdrawal symptoms.

Abuse-resistant, sustained-release dosage forms of products intended to treat pain have been described in the art (see, for example, U.S. Application Nos. 2003/0124185 and 2003/0044458). However, it is believed that substantial amounts of the opioid antagonist or other antagonist found in these sequestered forms are released over time (usually less than 24 hours) due to the osmotic pressure that builds up in the core of the sequestered form, as water permeates through the sequestered form into the core. The high osmotic pressure inside the core of the sequestered form causes the opioid antagonist or antagonist to be pushed out of the sequestered form, thereby causing the opioid antagonist or antagonist to be released from the sequestered form. As shown below, certain embodiments described herein provide improved forms of sequestered opioid antagonists and controlled-release opioid agonists.

In view of the foregoing drawbacks of the sequestered forms of the prior art, there exists a need in the art for methods of treating pain a sequestered form of an opioid antagonist or other antagonist that is not substantially released from the sequestered form due to osmotic pressure. The invention provides such a sequestering form of an opioid antagonist or antagonist. This and other objects and advantages of the invention, as well as additional inventive features, will be apparent from the description of the invention provided herein.

### BRIEF DESCRIPTION OF THE DRAWINGS

FIG. 1. Abuse Liability Study: Summary of Primary Endpoint.

FIG. 2. Abuse Liability Study: Summary of Secondary Endpoint.

### BRIEF SUMMARY OF THE INVENTION

This invention pertains to compositions and methods useful for treating pain in human patients. One such composition contains both an opioid antagonist and an opioid agonist formulated such that the agonist is released over time with minimal release of the antagonist. Also provided are optimal ratios at which an opioid and an opioid antagonist may be combined for administration to humans such that the opioid activity is inhibited. These ratios may also be used to formulate compositions containing both an opioid and an opioid antagonist within a single pharmaceutical dosing unit.

### DETAILED DESCRIPTION OF THE INVENTION

Provided herein are compositions and methods for administering a multiple active agents to a mammal in a form and manner that minimizes the effects of either active agent upon the other in vivo. In certain embodiments, at least two active agents are formulated as part of a pharmaceutical composition. A first active agent may provide a therapeutic effect in vivo. The second active agent may be an antagonist of the first active agent, and may be useful in preventing misuse of the composition. For instance, where the first active agent is a narcotic, the second active agent may be an antagonist of the narcotic. The composition remains intact during normal usage by patients and the antagonist is not released. However, upon tampering with the composition, the antagonist may be released thereby preventing the narcotic from having its intended effect. In certain embodiments, the active agents are both contained within a single unit, such as a bead, in the form of layers. The active agents may be formulated with a substantially impermeable barrier as, for example, a controlled-release composition, such that release of the antagonist from the composition is minimized. In certain embodiments, the antagonist is released in in vitro assays but is substantially not released in vivo. In vitro and in vivo release of the active agent from the composition may be measured by any of several well-known techniques. For instance, in vivo release may be determined by measuring the plasma levels of the active agent or metabolites thereof (i.e., AUC, Cmax).

In certain embodiments, one of the active agents is an opioid receptor agonist. Several opioid agonists are commercially available or in clinical trials and may be administered as described herein such that the alcohol effects are minimized. Opioid agonists include, for example, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenazocine, phenomorphan, phenoperidine, piminodine, piritramide, propheptazine, promedol, properidine, propiram, propoxyphene, sufentanil, tramadol, tilidine, derivatives or complexes thereof, pharmaceutically acceptable salts thereof, and combinations thereof. Preferably, the opioid agonist is selected from the group consisting of hydrocodone, hydromorphone, oxycodone, dihydrocodeine, codeine, dihydromorphine, morphine, buprenorphine, derivatives or complexes thereof, pharmaceutically acceptable salts thereof, and combinations thereof. Most preferably, the opioid agonist is morphine, hydromorphone, oxycodone or hydrocodone. Equianalgesic doses of these opioids, in comparison to a 15 mg dose of hydrocodone, are as follows: oxycodone (13.5 mg), codeine (90.0 mg), hydrocodone (15.0 mg), hydromorphone (3.375 mg), levorphanol (1.8 mg), meperidine (135.0 mg), methadone (9.0 mg), and morphine (27.0 mg).

A common dosage form of hydrocodone is in combination with acetaminophen and is, commercially available, for example, as Lortab) in the United States from UCB Pharma, Inc. (Brussels, Belgium), as 2.5/500 mg, 5/500 mg, 7.5/500 mg and 10/500 mg hydrocodone/acetaminophen tablets. Tablets are also available in the ratio of 7.5 mg hydrocodone bitartrate and 650 mg acetaminophen and a 7.5 mg hydrocodone bitartrate and 750 mg acetaminophen. Hydrocodone, in combination with aspirin, is given in an oral dosage form to adults generally in 1-2 tablets every 4-6 hours as needed to alleviate pain. The tablet form is 5 mg hydrocodone bitartrate and 224 mg aspirin with 32 mg caffeine; or 5 mg hydrocodone bitartrate and 500 mg aspirin. Another formulation comprises hydrocodone bitartrate and ibuprofen. Vicoprofen®, commercially available in the U.S. from Knoll Laboratories (Mount Olive, N.J.), is a tablet containing 7.5 mg hydrocodone bitartrate and 200 mg ibuprofen. The invention is contemplated to encompass all such formulations, with the inclusion of the opioid antagonist and/or antagonist in sequestered form as part of a subunit comprising an opioid agonist.

Oxycodone, chemically known as 4,5-epoxy-14-hydroxy-3-methoxy-17-methylmorphinan-6-one, is an opioid agonist whose principal therapeutic action is analgesia. Other therapeutic effects of oxycodone include anxiolysis, euphoria and feelings of relaxation. The precise mechanism of its analgesic action is not known, but specific CNS opioid receptors for endogenous compounds with opioid-like activity have been identified throughout the brain and spinal cord and play a role in the analgesic effects of this drug. Oxycodone is commercially available in the United States, e.g., as Oxycotin® from Purdue Pharma L.P. (Stamford, Conn.), as controlled-release tablets for oral administration containing 10 mg, 20 mg, 40 mg or 80 mg oxycodone hydrochloride, and as OxyIR™, also from Purdue Pharma L.P., as immediate-release capsules containing 5 mg oxycodone hydrochloride. The invention is contemplated to encompass all such formulations, with the inclusion of an opioid antagonist and/or antagonist in sequestered form as part of a subunit comprising an opioid agonist.

Oral hydromorphone is commercially available in the United States, e.g., as Dilaudid® from Abbott Laboratories (Chicago, Ill.). Oral morphine is commercially available in the United States, e.g., as Kadian® from Faulding Laboratories (Piscataway, N.J.).

In embodiments in which the opioid agonist comprises hydrocodone, the sustained-release oral dosage forms can include analgesic doses from about 8 mg to about 50 mg of hydrocodone per dosage unit. In sustained-release oral dosage forms where hydromorphone is the therapeutically active opioid, it is included in an amount from about 2 mg to about 64 mg hydromorphone hydrochloride. In another embodiment, the opioid agonist comprises morphine, and the sustained-release oral dosage forms of the invention include from about 2.5 mg to about 800 mg morphine, by weight. In yet another embodiment, the opioid agonist comprises oxycodone and the sustained-release oral dosage forms include from about 2.5 mg to about 800 mg oxycodone. In certain preferred embodiments, the sustained-release oral dosage forms include from about 20 mg to about 30 mg oxycodone. Controlled release oxycodone formulations are known in the art. The following documents describe various controlled-release oxycodone formulations suitable for use in the invention described herein, and processes for their manufacture: U.S. Pat. Nos. 5,266,331; 5,549,912; 5,508,042; and 5,656,295, which are incorporated herein by reference. The opioid agonist can comprise tramadol and the sustained-release oral dosage forms can include from about 25 mg to 800 mg tramadol per dosage unit.

In certain embodiments, another active agent contained within the composition may be an opioid receptor antagonist. In certain embodiments, the agonist and antagonist are administered together, either separately or as part of a single pharmaceutical unit. In the instance when the therapeutic agent is an opioid agonist, the antagonist preferably is an opioid antagonist, such as naltrexone, naloxone, nalmefene, cyclazacine, levallorphan, derivatives or complexes thereof, pharmaceutically acceptable salts thereof, and combinations thereof. More preferably, the opioid antagonist is naloxone or naltrexone. By “opioid antagonist” is meant to include one or more opioid antagonists, either alone or in combination, and is further meant to include partial antagonists, pharmaceutically acceptable salts thereof, stereoisomers thereof, ethers thereof, esters thereof, and combinations thereof. The pharmaceutically acceptable salts include metal salts, such as sodium salt, potassium salt, cesium salt, and the like; alkaline earth metals, such as calcium salt, magnesium salt, and the like; organic amine salts, such as triethylamine salt, pyridine salt, picoline salt, ethanolamine salt, triethanolamine salt, dicyclohexylamine salt, N,N-dibenzylethylenediamine salt, and the like; inorganic acid salts, such as hydrochloride, hydrobromide, sulfate, phosphate, and the like; organic acid salts, such as formate, acetate, trifluoroacetate, maleate, tartrate, and the like; sulfonates, such as methanesulfonate, benzenesulfonate, p-toluenesulfonate, and the like; amino acid salts, such as arginate, asparginate, glutamate, and the like. In certain embodiments, the amount of the opioid antagonist can be about 10 ng to about 275 mg. In a preferred embodiment, when the antagonist is naltrexone, it is preferable that the intact dosage form releases less than 0.125 mg or less within 24 hours, with 0.25 mg or greater of naltrexone released after 1 hour when the dosage form is crushed or chewed.

In a preferred embodiment, the opioid antagonist comprises naloxone. Naloxone is an opioid antagonist, which is almost void of agonist effects. Subcutaneous doses of up to 12 mg of naloxone produce no discernable subjective effects, and 24 mg naloxone causes only slight drowsiness. Small doses (0.4-0.8 mg) of naloxone given intramuscularly or intravenously in man prevent or promptly reverse the effects of morphine-like opioid agonist. One mg of naloxone intravenously has been reported to block completely the effect of 25 mg of heroin. The effects of naloxone are seen almost immediately after intravenous administration. The drug is absorbed after oral administration, but has been reported to be metabolized into an inactive form rapidly in its first passage through the liver, such that it has been reported to have significantly lower potency than when parenterally administered. Oral dosages of more than 1 g have been reported to be almost completely metabolized in less than 24 hours. It has been reported that 25% of naloxone administered sublingually is absorbed (Weinberg et al., Clin. Pharmacol. Ther. 44:335-340 (1988)).

In another preferred embodiment, the opioid antagonist comprises naltrexone. In the treatment of patients previously addicted to opioids, naltrexone has been used in large oral doses (over 100 mg) to prevent euphorigenic effects of opioid agonists. Naltrexone has been reported to exert strong preferential blocking action against mu over delta sites. Naltrexone is known as a synthetic congener of oxymorphone with no opioid agonist properties, and differs in structure from oxymorphone by the replacement of the methyl group located on the nitrogen atom of oxymorphone with a cyclopropylmethyl group. The hydrochloride salt of naltrexone is soluble in water up to about 100 mg/cc. The pharmacological and pharmacokinetic properties of naltrexone have been evaluated in multiple animal and clinical studies. See, e.g., Gonzalez et al. Drugs 35:192-213 (1988). Following oral administration, naltrexone is rapidly absorbed (within 1 hour) and has an oral bioavailability ranging from 5-40%. Naltrexone's protein binding is approximately 21% and the volume of distribution following single-dose administration is 16.1 L/kg.

Naltrexone is commercially available in tablet form (Revia®, DuPont (Wilmington, Del.)) for the treatment of alcohol dependence and for the blockade of exogenously administered opioids. See, e.g., Revia (naltrexone hydrochloride tablets), Physician's Desk Reference, 51^{st} ed., Montvale, N.J.; and Medical Economics 51:957-959 (1997). A dosage of 50 mg Revia® blocks the pharmacological effects of 25 mg IV administered heroin for up to 24 hours. It is known that, when coadministered with morphine, heroin or other opioids on a chronic basis, naltrexone blocks the development of physical dependence to opioids. It is believed that the method by which naltrexone blocks the effects of heroin is by competitively binding at the opioid receptors. Naltrexone has been used to treat narcotic addiction by complete blockade of the effects of opioids. It has been found that the most successful use of naltrexone for a narcotic addiction is with narcotic addicts having good prognosis, as part of a comprehensive occupational or rehabilitative program involving behavioral control or other compliance-enhancing methods. For treatment of narcotic dependence with naltrexone, it is desirable that the patient be opioid-free for at least 7-10 days. The initial dosage of naltrexone for such purposes has typically been about 25 mg, and if no withdrawal signs occur, the dosage may be increased to 50 mg per day. A daily dosage of 50 mg is considered to produce adequate clinical blockade of the actions of parenterally administered opioids. Naltrexone also has been used for the treatment of alcoholism as an adjunct with social and psychotherapeutic methods.

Other preferred opioid antagonists include, for example, cyclazocine and naltrexone, both of which have cyclopropylmethyl substitutions on the nitrogen, retain much of their efficacy by the oral route, and last longer, with durations approaching 24 hours after oral administration.

The antagonist may also be a bittering agent. The term “bittering agent” as used herein refers to any agent that provides an unpleasant taste to the host upon inhalation and/or swallowing of a tampered dosage form comprising the sequestering subunit. With the inclusion of a bittering agent, the intake of the tampered dosage form produces a bitter taste upon inhalation or oral administration, which, in certain embodiments, spoils or hinders the pleasure of obtaining a high from the tampered dosage form, and preferably prevents the abuse of the dosage form.

Various bittering agents can be employed including, for example, and without limitation, natural, artificial and synthetic flavor oils and flavoring aromatics and/or oils, oleoresins and extracts derived from plants, leaves, flowers, fruits, and so forth, and combinations thereof. Nonlimiting representative flavor oils include spearmint oil, peppermint oil, eucalyptus oil, oil of nutmeg, allspice, mace, oil of bitter almonds, menthol and the like. Also useful bittering agents are artificial, natural and synthetic fruit flavors such as citrus oils, including lemon, orange, lime, and grapefruit, fruit essences, and so forth. Additional bittering agents include sucrose derivatives (e.g., sucrose octaacetate), chlorosucrose derivatives, quinine sulphate, and the like. A preferred bittering agent for use in the invention is Denatonium Benzoate NF-Anhydrous, sold under the name Bitrex™ (Macfarlan Smith Limited, Edinburgh, UK). A bittering agent can be added to the formulation in an amount of less than about 50% by weight, preferably less than about 10% by weight, more preferably less than about 5% by weight of the dosage form, and most preferably in an amount ranging from about 0.1 to 1.0 percent by weight of the dosage form, depending on the particular bittering agent(s) used.

Alternatively, the antagonist may be a dye. The term “dye” as used herein refers to any agent that causes discoloration of the tissue in contact. In this regard, if the sequestering subunit is tampered with and the contents are snorted, the dye will discolor the nasal tissues and surrounding tissues thereof. Preferred dyes are those that can bind strongly with subcutaneous tissue proteins and are well-known in the art. Dyes useful in applications ranging from, for example, food coloring to tattooing, are exemplary dyes suitable for the invention. Food coloring dyes include, but are not limited to FD&C Green #3 and FD&C Blue #1, as well as any other FD&C or D&C color. Such food dyes are commercially available through companies, such as Voigt Global Distribution (Kansas City, Mo.).

The antagonist may alternatively be an irritant. The term “irritant” as used herein includes a compound used to impart an irritating, e.g., burning or uncomfortable, sensation to an abuser administering a tampered dosage form of the invention. Use of an irritant will discourage an abuser from tampering with the dosage form and thereafter inhaling, injecting, or swallowing the tampered dosage form. Preferably, the irritant is released when the dosage form is tampered with and provides a burning or irritating effect to the abuser upon inhalation, injection, and/or swallowing the tampered dosage form. Various irritants can be employed including, for example, and without limitation, capsaicin, a capsaicin analog with similar type properties as capsaicin, and the like. Some capsaicin analogues or derivatives include, for example, and without limitation, resiniferatoxin, tinyatoxin, heptanoylisobutylamide, heptanoyl guaiacylamide, other isobutylamides or guaiacylamides, dihydrocapsaicin, homovanillyl octylester, nonanoyl vanillylamide, or other compounds of the class known as vanilloids. Resiniferatoxin is described, for example, in U.S. Pat. No. 5,290,816. U.S. Pat. No. 4,812,446 describes capsaicin analogs and methods for their preparation. Furthermore, U.S. Pat. No. 4,424,205 cites Newman, “Natural and Synthetic Pepper-Flavored Substances,” published in 1954 as listing pungency of capsaicin-like analogs. Ton et al., British Journal of Pharmacology 10:175-182 (1955), discusses pharmacological actions of capsaicin and its analogs. With the inclusion of an irritant (e.g., capsaicin) in the dosage form, the irritant imparts a burning or discomforting quality to the abuser to discourage the inhalation, injection, or oral administration of the tampered dosage form, and preferably to prevent the abuse of the dosage form. Suitable capsaicin compositions include capsaicin (trans 8-methyl-N-vanillyl-6-noneamide) or analogues thereof in a concentration between about 0.00125% and 50% by weight, preferably between about 1% and about 7.5% by weight, and most preferably, between about 1% and about 5% by weight:

The antagonist may also be a gelling agent. The term “gelling agent” as used herein refers to any agent that provides a gel-like quality to the tampered dosage form, which slows the absorption of the therapeutic agent, which is formulated with the sequestering subunit, such that a host is less likely to obtain a rapid “high.” In certain preferred embodiments, when the dosage form is tampered with and exposed to a small amount (e.g., less than about 10 ml) of an aqueous liquid (e.g., water), the dosage form will be unsuitable for injection and/or inhalation. Upon the addition of the aqueous liquid, the tampered dosage form preferably becomes thick and viscous, rendering it unsuitable for injection. The term “unsuitable for injection” is defined for purposes of the invention to mean that one would have substantial difficulty injecting the dosage form (e.g., due to pain upon administration or difficulty pushing the dosage form through a syringe) due to the viscosity imparted on the dosage form, thereby reducing the potential for abuse of the therapeutic agent in the dosage form. In certain embodiments, the gelling agent is present in such an amount in the dosage form that attempts at evaporation (by the application of heat) to an aqueous mixture of the dosage form in an effort to produce a higher concentration of the therapeutic agent, produces a highly viscous substance unsuitable for injection. When nasally inhaling the tampered dosage form, the gelling agent can become gel-like upon administration to the nasal passages, due to the moisture of the mucous membranes. This also makes such formulations aversive to nasal administration, as the gel will stick to the nasal passage and minimize absorption of the abusable substance. Various gelling agents may can be employed including, for example, and without limitation, sugars or sugar-derived alcohols, such as mannitol, sorbitol, and the like, starch and starch derivatives, cellulose derivatives, such as microcrystalline cellulose, sodium caboxymethyl cellulose, methylcellulose, ethyl cellulose, hydroxyethyl cellulose, hydroxypropyl cellulose, and hydroxypropyl methylcellulose, attapulgites, bentonites, dextrins, alginates, carrageenan, gum tragacant, gum acacia, guar gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and mixtures thereof; etc. In certain preferred embodiments, the gelling agent is xanthan gum. In other preferred embodiments, the gelling agent of the invention is pectin. The pectin or pectic substances useful for this invention include not only purified or isolated pectates but also crude natural pectin sources, such as apple, citrus or sugar beet residues, which have been subjected, when necessary, to esterification or de-esterification, e.g., by alkali or enzymes. Preferably, the pectins used in this invention are derived from citrus fruits, such as lime, lemon, grapefruit, and orange. With the inclusion of a gelling agent in the dosage form, the gelling agent preferably imparts a gel-like quality to the dosage form upon tampering that spoils or hinders the pleasure of obtaining a rapid high from due to the gel-like consistency of the tampered dosage form in contact with the mucous membrane, and in certain embodiments; prevents the abuse of the dosage form by minimizing absorption, e.g., in the nasal passages. A gelling agent can be added to the formulation in a ratio of gelling agent to opioid agonist of from about 1:40 to about 40:1 by weight, preferably from about 1:1 to about 30:1 by weight, and more preferably from about 2:1 to about 10:1 by weight of the opioid agonist. In certain other embodiments, the dosage form forms a viscous gel having a viscosity of at least about 10 cP after the dosage form is tampered with by dissolution in an aqueous liquid (from about 0.5 to about 10 ml and preferably from 1 to about 5 ml). Most preferably, the resulting mixture will have a viscosity of at least about 60 cP.

The antagonist can comprise a single type of antagonist (e.g., a capsaicin), multiple forms of a single type of antagonist (e.g., a capasin and an analogue thereof), or a combination of different types of antagonists (e.g., one or more bittering agents and one or more gelling agents). Desirably, the amount of antagonist in a unit of the invention is not toxic to the host.

In one embodiment, the invention provides a sequestering subunit comprising an opioid antagonist and a blocking agent, wherein the blocking agent substantially prevents release of the opioid antagonist from the sequestering subunit in the gastrointestinal tract for a time period that is greater than 24 hours. This sequestering subunit is incorporated into a single pharmaceutical unit that also includes an opioid agonist. The pharmaceutical unit thus includes a core portion to which the opioid antagonist is applied. A seal coat is then optionally applied upon the antagonist. Upon the seal coat is then applied a composition comprising the pharmaceutically active agent. An additional layer containing the same or a different blocking agent may then be applied such that the opioid agonist is released in the digestive tract over time (i.e., controlled release). Thus, the opioid antagonist and the opioid agonist are both contained within a single pharmaceutical unit, which is typically in the form of a bead.

The term “sequestering subunit” as used herein refers to any means for containing an antagonist and preventing or substantially preventing the release thereof in the gastrointestinal tract when intact, i.e., when not tampered with. The term “blocking agent” as used herein refers to the means by which the sequestering subunit is able to prevent substantially the antagonist from being released. The blocking agent may be a sequestering polymer, for instance, as described in greater detail below.

The terms “substantially prevents,” “prevents,” or any words stemming therefrom, as used herein, means that the antagonist is substantially not released from the sequestering subunit in the gastrointestinal tract. By “substantially not released” is meant that the antagonist may be released in a small amount, but the amount released does not affect or does not significantly affect the analgesic efficacy when the dosage form is orally administered to a host, e.g., a mammal (e.g., a human), as intended. The terms “substantially prevents,” “prevents,” or any words stemming therefrom, as used herein, does not necessarily imply a complete or 100% prevention. Rather, there are varying degrees of prevention of which one of ordinary skill in the art recognizes as having a potential benefit. In this regard, the blocking agent substantially prevents or prevents the release of the antagonist to the extent that at least about 80% of the antagonist is prevented from being released from the sequestering subunit in the gastrointestinal tract for a time period that is greater than 24 hours. Preferably, the blocking agent prevents release of at least about 90% of the antagonist from the sequestering subunit in the gastrointestinal tract for a time period that is greater than 24 hours. More preferably, the blocking agent prevents release of at least about 95% of the antagonist from the sequestering subunit. Most preferably, the blocking agent prevents release of at least about 99% of the antagonist from the sequestering subunit in the gastrointestinal tract for a time period that is greater than 24 hours.

For purposes of this invention, the amount of the antagonist released after oral administration can be measured in-vitro by dissolution testing as described in the United States Pharmacopeia (USP26) in chapter <711> Dissolution. For example, using 900 mL of 0.1 N HCl, Apparatus 2 (Paddle), 75 rpm, at 37° C. to measure release at various times from the dosage unit. Other methods of measuring the release of an antagonist from a sequestering subunit over a given period of time are known in the art (see, e.g., USP26).

Without being bound to any particular theory, it is believed that the sequestering subunit of the invention overcomes the limitations of the sequestered forms of an antagonist known in the art in that the sequestering subunit of the invention reduces osmotically-driven release of the antagonist from the sequestering subunit. Furthermore, it is believed that the present inventive sequestering subunit reduces the release of the antagonist for a longer period of time (e.g., greater than 24 hours) in comparison to the sequestered forms of antagonists known in the art. The fact that the sequestered subunit of the invention provides a longer prevention of release of the antagonist is particularly relevant, since precipitated withdrawal could occur after the time for which the therapeutic agent is released and acts. It is well known that the gastrointestinal tract transit time for individuals varies greatly within the population. Hence, the residue of the dosage form may be retained in the tract for longer than 24 hours, and in some cases for longer than 48 hours. It is further well known that opioid analgesics cause decreased bowel motility, further prolonging gastrointestinal tract transit time. Currently, sustained-release forms having an effect over a 24 hour time period have been approved by the Food and Drug Administration. In this regard, the present inventive sequestering subunit provides prevention of release of the antagonist for a time period that is greater than 24 hours when the sequestering subunit has not been tampered.

The sequestering subunit of the invention is designed to prevent substantially the release of the antagonist when intact. By “intact” is meant that a dosage form has not undergone tampering. The term “tampering” is meant to include any manipulation by mechanical, thermal and/or chemical means, which changes the physical properties of the dosage form. The tampering can be, for example, crushing, shearing, grinding, chewing, dissolution in a solvent, heating (for example, greater than about 45° C.), or any combination thereof. When the sequestering subunit of the invention has been tampered with, the antagonist is immediately released from the sequestering subunit.

By “subunit” is meant to include a composition, mixture, particle; etc., that can provide a dosage form (e.g., an oral dosage form) when combined with another subunit. The subunit can be in the form of a bead, pellet, granule, spheroid, or the like, and can be combined with additional same or different subunits, in the form of a capsule, tablet or the like, to provide a dosage form, e.g., an oral dosage form. The subunit may also be part of a larger, single unit, forming part of that unit, such as a layer. For instance, the subunit may be a core coated with an antagonist and a seal coat; this subunit may then be coated with additional compositions including a pharmaceutically active agent such as an opioid agonist.

For purposes of the invention; the antagonist can be any agent that negates the effect of the therapeutic agent or produces an unpleasant or punishing stimulus or effect, which will deter or cause avoidance of tampering with the sequestering subunit or compositions comprising the same. Desirably, the antagonist does not harm a host by its administration or consumption but has properties that deter its administration or consumption, e.g., by chewing and swallowing or by crushing and snorting, for example. The antagonist can have a strong or foul taste or smell, provide a burning or tingling sensation, cause a lachrymation response, nausea, vomiting, or any other unpleasant or repugnant sensation, or color tissue, for example. Preferably, the antagonist is selected from the group consisting of an antagonist of a therapeutic agent, a bittering agent, a dye, a gelling agent, and an irritant. Exemplary antagonists include capsaicin, dye, bittering agents and emetics.

By “antagonist of a therapeutic agent” is meant any drug or molecule, naturally-occurring or synthetic, that binds to the same target molecule (e.g., a receptor) of the therapeutic agent, yet does not produce a therapeutic, intracellular, or in vivo response. In this regard, the antagonist of a therapeutic agent binds to the receptor of the therapeutic agent, thereby preventing the therapeutic agent from acting on the receptor, thereby preventing the achievement of a “high” in the host.

In the instance when the therapeutic agent is an opioid agonist, the antagonist preferably is an opioid antagonist, such as naltrexone, naloxone, nalmefene, cyclazacine, levallorphan, derivatives or complexes thereof pharmaceutically acceptable salts thereof, and combinations thereof. More preferably, the opioid antagonist is naloxone or naltrexone. By “opioid antagonist” is meant to include one or more opioid antagonists, either alone or in combination, and is further meant to include partial antagonists, pharmaceutically acceptable salts thereof, stereoisomers thereof, ethers thereof, esters thereof, and combinations thereof. The pharmaceutically acceptable salts include metal salts, such as sodium salt, potassium salt, cesium salt, and the like; alkaline earth metals, such as calcium salt, magnesium salt, and the like; organic amine salts, such as triethylamine salt, pyridine salt, picoline salt, ethanolamine salt, triethanolamine salt, dicyclohexylamine salt, N,N-dibenzylethylenediamine salt, and the like; inorganic acid salts, such as hydrochloride, hydrobromide, sulfate, phosphate, and the like; organic acid salts, such as formate, acetate, trifluoroacetate, maleate, tartrate, and the like; sulfonates, such as methanesulfonate, benzenesulfonate, p-toluenesulfonate, and the like; amino acid salts, such as arginate, asparginate, glutamate, and the like. In certain embodiments, the amount of the opioid antagonist, present in sequestered form, can be about 10 ng to about 275 mg. In a preferred embodiment, when the antagonist is naltrexone, it is preferable that the intact dosage form releases less than 0.125 mg or less within 24 hours, with 0.25 mg or greater of naltrexone released after 1 hour when the dosage form is crushed or chewed.

The antagonist can comprise a single type of antagonist (e.g., a capsaicin), multiple forms of a single type of antagonist (e.g., a capasin and an analogue thereof), or a combination of different types of antagonists (e.g., one or more bittering agents and one or more gelling agents). Desirably, the amount of antagonist in the sequestering subunit of the invention is not toxic to the host.

The blocking agent prevents or substantially prevents the release of the antagonist in the gastrointestinal tract for a time period that is greater than 24 hours, e.g., between 24 and 25 hours, 30 hours, 35 hours, 40 hours, 45 hours, 48 hours, 50 hours, 55 hours, 60 hours, 65 hours, 70 hours, 72 hours, 75 hours, 80 hours, 85 hours, 90 hours, 95 hours, or 100 hours; etc. Preferably, the time period for which the release of the antagonist is prevented or substantially prevented in the gastrointestinal tract is at least about 48 hours. More preferably, the blocking agent prevents or substantially prevents the release for a time period of at least about 72 hours.

The blocking agent of the present inventive sequestering subunit can be a system comprising a first antagonist-impermeable material and a core. By “antagonist-impermeable material” is meant any material that is substantially impermeable to the antagonist, such that the antagonist is substantially not released from the sequestering subunit. The term “substantially impermeable” as used herein does not necessarily imply complete or 100% impermeability. Rather, there are varying degrees of impermeability of which one of ordinary skill in the art recognizes as having a potential benefit. In this regard, the antagonist-impermeable material substantially prevents or prevents the release of the antagonist to an extent that at least about 80% of the antagonist is prevented from being released from the sequestering subunit in the gastrointestinal tract for a time period that is greater than 24 hours. Preferably, the antagonist-impermeable material prevents release of at least about 90% of the antagonist from the sequestering subunit in the gastrointestinal tract for a time period that is greater than 24 hours. More preferably, the antagonist-impermeable material prevents release of at least about 95% of the antagonist from the sequestering subunit. Most preferably, the antagonist-impermeable material prevents release of at least about 99% of the antagonist from the sequestering subunit in the gastrointestinal tract for a time period that is greater than 24 hours. The antagonist-impermeable material prevents or substantially prevents the release of the antagonist in the gastrointestinal tract for a time period that is greater than 24 hours, and desirably, at least about 48 hours. More desirably, the antagonist-impermeable material prevents or substantially prevents the release of the adversive agent from the sequestering subunit for a time period of at least about 72 hours.

Preferably, the first antagonist-impermeable material comprises a hydrophobic material, such that the antagonist is not released or substantially not released during its transit through the gastrointestinal tract when administered orally as intended, without having been tampered with. Suitable hydrophobic materials for use in the invention are described herein and set forth below. The hydrophobic material is preferably a pharmaceutically acceptable hydrophobic material. Preferably, the pharmaceutically acceptable hydrophobic material comprises a cellulose polymer.

It is preferred that the first antagonist-impermeable material comprises a polymer insoluble in the gastrointestinal tract. One of ordinary skill in the art appreciates that a polymer that is insoluble in the gastrointestinal tract will prevent the release of the antagonist upon ingestion of the sequestering subunit. The polymer can be a cellulose or an acrylic polymer. Desirably, the cellulose is selected from the group consisting of ethylcellulose, cellulose acetate, cellulose propionate, cellulose acetate propionate, cellulose acetate butyrate, cellulose acetate phthalate, cellulose triacetate, and combinations thereof. Ethylcellulose includes, for example, one that has an ethoxy content of about 44 to about 55%. Ethylcellulose can be used in the form of an aqueous dispersion, an alcoholic solution, or a solution in other suitable solvents. The cellulose can have a degree of substitution (D.S.) on the anhydroglucose unit, from greater than zero and up to 3 inclusive. By “degree of substitution” is meant the average number of hydroxyl groups on the anhydroglucose unit of the cellulose polymer that are replaced by a substituting group. Representative materials include a polymer selected from the group consisting of cellulose acylate, cellulose diacylate, cellulose triacylate, cellulose acetate, cellulose diacetate, cellulose triacetate, monocellulose alkanylate, dicellulose alkanylate, tricellulose alkanylate, monocellulose alkenylates, dicellulose alkenylates, tricellulose alkenylates, monocellulose aroylates, dicellulose aroylates, and tricellulose aroylates.

More specific celluloses include cellulose propionate having a D.S. of 1.8 and a propyl content of 39.2 to 45 and a hydroxy content of 2.8 to 5.4%; cellulose acetate butyrate having a D.S. of 1.8, an acetyl content of 13 to 15% and a butyryl content of 34 to 39%; cellulose acetate butyrate having an acetyl content of 2 to 29%, a butyryl content of 17 to 53% and a hydroxy content of 0.5 to 4.7%; cellulose triacylate having a D.S. of 2.9 to 3, such as cellulose triacetate, cellulose trivalerate, cellulose trilaurate, cellulose tripatmitate, cellulose trisuccinate, and cellulose trioctanoate; cellulose diacylates having a D.S. of 2.2 to 2.6, such as cellulose disuccinate, cellulose dipalmitate, cellulose dioctanoate, cellulose dipentanoate, and coesters of cellulose, such as cellulose acetate butyrate, cellulose acetate octanoate butyrate, and cellulose acetate propionate.

Additional cellulose polymers useful for preparing a sequestering subunit of the invention includes acetaldehyde dimethyl cellulose acetate, cellulose acetate ethylcarbamate, cellulose acetate methycarbamate, and cellulose acetate dimethylaminocellulose acetate.

The acrylic polymer preferably is selected from the group consisting of methacrylic polymers, acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers, and combinations thereof. An acrylic polymer useful for preparation of a sequestering subunit of the invention includes acrylic resins comprising copolymers synthesized from acrylic and methacrylic acid esters (e.g., the copolymer of acrylic acid lower alkyl ester and methacrylic acid lower alkyl ester) containing about 0.02 to about 0.03 mole of a tri (lower alkyl) ammonium group per mole of the acrylic and methacrylic monomer used. An example of a suitable acrylic resin is ammonio methacrylate copolymer NF21, a polymer manufactured by Rohm Fharma GmbH, Darmstadt, Germany, and sold under the Eudragit® trademark. Eudragit RS30D is preferred. Eudragit® is a water-insoluble copolymer of ethyl acrylate (EA), methyl methacrylate (MM) and trimethylammoniumethyl methacrylate chloride (TAM) in which the molar ratio of TAM to the remaining components (EA and MM) is 1:40. Acrylic resins, such as Eudragit®, can be used in the form of an aqueous dispersion or as a solution in suitable solvents.

In another preferred embodiment, the antagonist-impermeable material is selected from the group consisting of polylactic acid, polyglycolic acid, a co-polymer of polylactic acid and polyglycolic acid, and combinations thereof. In certain other embodiments, the hydrophobic material includes a biodegradable polymer comprising a poly(lactic/glycolic acid) (“PLGA”), a polylactide, a polyglycolide, a polyanhydride, a polyorthoester, polycaprolactones, polyphosphazenes, polysaccharides, proteinaceous polymers, polyesters, polydioxanone, polygluconate, polylactic-acid-polyethylene oxide copolymers, poly(hydroxybutyrate), polyphosphoester or combinations thereof.

Preferably, the biodegradable polymer comprises a poly(lactic/glycolic acid), a copolymer of lactic and glycolic acid, having a molecular weight of about 2,000 to about 500,000 daltons. The ratio of lactic acid to glycolic acid is preferably from about 100:1 to about 25:75, with the ratio of lactic acid to glycolic acid of about 65:35 being more preferred. Poly(lactic/glycolic acid) can be prepared by the procedures set forth in U.S. Pat. No. 4,293,539 (Ludwig et al.), which is incorporated herein by reference. In brief, Ludwig prepares the copolymer by condensation of lactic acid and glycolic acid in the presence of a readily removable polymerization catalyst (e.g., a strong ion-exchange resin such as Dowex HCR-W2-H). The amount of catalyst is not critical to the polymerization, but typically is from about 0.01 to about 20 parts by weight relative to the total weight of combined lactic acid and glycolic acid. The polymerization reaction can be conducted without solvents at a temperature from about 100° C. to about 250° C. for about 48 to about 96 hours, preferably under a reduced pressure to facilitate removal of water and by-products. Poly(lactic/glycolic acid) is then recovered by filtering the molten reaction mixture in an organic solvent, such as dichloromethane or acetone, and then filtering to remove the catalyst.

Suitable plasticizers, for example, acetyl triethyl citrate, acetyl tributyl citrate, triethyl citrate, diethyl phthalate, dibutyl phthalate, or dibutyl sebacate, also can be admixed with the polymer used to make the sequestering subunit. Additives, such as coloring agents, talc and/or magnesium stearate, and other additives also can be used in making the present inventive sequestering subunit.

In certain embodiments, additives may be included in the compositions to improve the sequestering characteristics of the sequestering subunit. As described below, the ratio of additives or components with respect to other additives or components may be modified to enhance or delay improve sequestration of the agent contained within the subunit. Various amounts of a functional additive (i.e., a charge-neutralizing additive) may be included to vary the release of an antagonist, particularly where a water-soluble core (i.e., a sugar sphere) is utilized. For instance, it has been determined that the inclusion of a low amount of charge-neutralizing additive relative to sequestering polymer on a weight-by-weight basis may cause decreased release of the antagonist.

In certain embodiments, a surfactant may serve as a charge-neutralizing additive. Such neutralization may in certain embodiments reduce the swelling of the sequestering polymer by hydration of positively charged groups contained therein. Surfactants (ionic or non-ionic) may also be used in preparing the sequestering subunit. It is preferred that the surfactant be ionic. Suitable exemplary agents include, for example, alkylaryl sulphonates, alcohol sulphates, sulphosuccinates, suiphosuccinamates, sarcosinates or taurates and others. Additional examples include but are not limited to ethoxylated castor oil, benzalkonium chloride, polyglycolyzed glycerides, acetylated monoglycerides, sorbitan fatty acid esters, poloxamers, polyoxyethylene fatty acid esters, polyoxyethylene derivatives, monoglycerides or ethoxylated derivatives thereof, diglycerides or polyoxyethylene-derivatives thereof, sodium docusate, sodium lauryl sulfate, dioctyl sodium sulphosuccinate, sodium lauryl sarcosinate and sodium methyl cocoyl taurate, magnesium lauryl sulfate, triethanolamine, cetrimide, sucrose laurate and other sucrose esters, glucose (dextrose) esters, simethicone, ocoxynol, dioctyl sodiumsulfosuceinate, polyglycolyzed glycerides, sodiumdodecylbenzene sulfonate, dialkyl sodiumsulfosuccinate, fatty alcohols such as lauryl, cetyl, and steryl, glycerylesters, cholic acid or derivatives thereof, lecithins, and phospholipids. These agents are typically characterized as ionic (i.e., anionic or cationic) or nonionic. In certain embodiments described herein, an anionic surfactant such as sodium lauryl sulfate (SLS) is preferably used (U.S. Pat. No. 5,725,883; U.S. Pat. No. 7,201,920; EP 502642A1; Shokri, et al. Pharm. Sci. 2003. The effect of sodium lauryl sulphate on the release of diazepam from solid dispersions prepared by cogrinding technique. Wells, et al. Effect of Anionic Surfactants on the Release of Chlorpheniramine Maleate From an Inert, Heterogeneous Matrix. Drug Development and Industrial Pharmacy 18(2) (1992): 175-186. Rao, et al. “Effect of Sodium Lauryl Sulfate on the Release of Rifampicin from Guar Gum Matrix.” Indian Journal of Pharmaceutical Science (2000): 404-406; Knop, et al. Influence of surfactants of different charge and concentration on drug release from pellets coated with an aqueous dispersion of quaternary acrylic polymers. STP Pharma Sciences, Vol. 7, No. 6, (1997) 507-512). Other suitable agents are known in the art.

As shown herein, SLS is particularly useful in combination with Eudragit RS when the sequestering subunit is built upon a sugar sphere substrate. The inclusion of SLS at less than approximately 6.3% on a weight-to-weight basis relative to the sequestering polymer (i.e., Eudragit RS) may provide a charge neutralizing function (theoretically 20% and 41% neutralization, respectfully), and thereby significantly slow the release of the active agent encapsulated thereby (i.e., the antagonist naltrexone). Inclusion of more than approximately 6.3% SLS relative to the sequestering polymer appears to increase release of the antagonist from the sequestering subunit. With respect to SLS used in conjunction with Eudragit® RS, it is preferred that the SLS is present at approximately 1%, 2%, 3%, 4% or 5%, and typically less than 6% on a w/w basis relative to the sequestering polymer (i.e., Eudragit® RS). In preferred embodiments, SLS may be present at approximately 1.6% or approximately 3.3% relative to the sequestering polymer. As discussed above, many agents (i.e., surfactants) may substitute for SLS in the compositions disclosed herein.

Additionally useful agents include those that may physically block migration of the antagonist from the subunit and/or enhance the hydrophobicity of the barrier. One exemplary agent is talc, which is commonly used in pharmaceutical compositions (Pawar et al. Agglomeration of Ibuprofen With Talc by Novel Crystallo-Co-Agglomeration Technique. AAPS PharmSciTech. 2004; 5(4): article 55). As shown in the Examples, talc is especially useful where the sequestering subunit is built upon a sugar sphere core. Any form of talc may be used, so long as it does not detrimentally affect the function of the composition. Most talc results from the alteration of dolomite (CaMg(CO₃)₂ or magnesite (MgO) in the presence of excess dissolved silica (SiO₂) or by altering serpentine or quartzite. Talc may be include minerals such as tremolite (CaMg₃(SiO₃)₄), serpentine (3MgO.2SiO₂.2H₂O), anthophyllite (Mg₇.(OH)₂.(Si₄O₁₁)₂), magnesite, mica, chlorite, dolomite, the calcite form of calcium carbonate (CaCO₃), iron oxide, carbon, quartz, and/or manganese oxide. The presence of such impurities may be acceptable in the compositions described herein provided the function of the talc is maintained. It is preferred that that talc be USP grade. As mentioned above, the function of talc as described herein is to enhance the hydrophobicity and therefore the functionality of the sequestering polymer. Many substitutes for talc may be utilized in the compositions described herein as may be determined by one of skill in the art.

It has been determined that the ratio of talc to sequestering polymer may make a dramatic difference in the functionality of the compositions described herein. For instance, the Examples described below demonstrate that the talc to sequestering polymer ratio (w/w) is important with respect to compositions designed to prevent the release of naltrexone therefrom. It is shown therein that inclusion of an approximately equivalent amount (on a weight-by-weight basis) of talc and Eudragit® RS results in a very low naltrexone release profile. In contrast, significantly lower or higher both a lower (69% w/w) and a higher (151% w/w) talc:Eudragit® RS ratios result in increased release of naltrexone release. Thus, where talc and Eudragit® RS are utilized, it is preferred that talc is present at approximately 75%, 80%, 85%, 90%, 95%, 100%, 105%, 110%, 115%, 120% or 125% w/w relative to Eudragit® RS. As described above, the most beneficial ratio for other additives or components will vary and may be determined using standard experimental procedures.

In certain embodiments, such as where a water-soluble core is utilized, it is useful to include agents that may affect the osmotic pressure of the composition (i.e., an osmotic pressure regulating agent) (see, in general, WO 2005/046561 A2 and WO 2005/046649 A2 relating to Eudramode®). This agent is preferably applied to the Eudragit® RS/talc layer described above. In a pharmaceutical unit comprising a sequestering subunit overlayed by an active agent (i.e., a controlled-release agonist preparation), the osmotic pressure regulating agent is preferably positioned immediately beneath the active agent layer. Suitable osmotic pressure regulating agents may include, for instance, hydroxypropylmethyl cellulose (HPMC) or chloride ions (i.e., from NaCl), or a combination of HPMC and chloride ions (i.e., from NaCl). Other ions that may be useful include bromide or iodide. The combination of sodium chloride and HPMC may be prepared in water or in a mixture of ethanol and water, for instance. HPMC is commonly utilized in pharmaceutical compositions (see, for example, U.S. Pat. Nos. 7,226,620 and 7,229,982). In certain embodiments, HPMC may have a molecular weight ranging from about 10,000 to about 1,500,000, and typically from about 5000 to about 10,000 (low molecular weight HPMC). The specific gravity of HPMC is typically from about 1.19 to about 1.31, with an average specific gravity of about 1.26 and a viscosity of about 3600 to 5600. HPMC may be a water-soluble synthetic polymer. Examples of suitable, commercially available hydroxypropyl methylcellulose polymers include Methocel K100 LV and Methocel K4M (Dow). Other HPMC additives are known in the art and may be suitable in preparing the compositions described herein. As shown in the Examples, the inclusion of NaCl (with HPMC) was found to have positively affect sequestration of naltrexone by Eudragit® RS. In certain embodiments, it is preferred that the charge-neutralizing additive (i.e., NaCl) is included at less than approximately 1, 2, 3, 4, 5, 6, 7, 8, 9, or 10% of the composition on a weight-by-weight basis. In other preferred embodiments, the charge-neutralizing additive is present at approximately 4% of the composition on a weight-by-weight basis.

Thus, in one embodiment, a sequestering subunit built upon a sugar sphere substrate is provided comprising a sequestering polymer (i.e., Eudragit® RS) in combination with several optimizing agents, including sodium lauryl sulfate (SLS) as a charge-neutralizing agent to reduce swelling of the film by hydration of the positively charged groups on the polymer; talc to create a solid impermeable obstacle to naltrexone transport through the film and as a hydrophobicity-enhancing agent; and a chloride ion (i.e., as NaCl) as an osmotic pressure reducing agent. The ratio of each of the additional ingredients relative to the sequestering polymer was surprisingly found to be important to the function of the sequestering subunit. For instance, the Examples provide a sequestering subunit including a sequestering polymer and the optimizing agents SLS at less than 6%, preferably 1-4%, and even more preferably 1.6% or 3.3% on a w/w basis relative to Eudragit RS; talc in an amount approximately equal to Eudragit® RS (on a w/w basis); and, NaCl present at approximately 4% on a w/w basis relative to Eudragit® RS.

The therapeutic agent applied upon the sequestering subunit may be any medicament. The therapeutic agent of the present inventive compositions can be any medicinal agent used for the treatment of a condition or disease, a pharmaceutically acceptable salt thereof, or an analogue of either of the foregoing. The therapeutic agent can be, for example, an analgesic (e.g., an opioid agonist, aspirin, acetaminophen, non-steroidal anti-inflammatory drugs (“NSAIDS”), N-methyl-D-aspartate (“NMDA”) receptor antagonists, cycooxygenase-II inhibitors (“COX-II inhibitors”), and glycine receptor antagonists), an antibacterial agent, an anti-viral agent, an anti-microbial agent, anti-infective agent, a chemotherapeutic, an immunosuppressant agent, an antitussive, an expectorant, a decongestant, an antihistamine drugs, a decongestant, antihistamine drugs, and the like. Preferably, the therapeutic agent is one that is addictive (physically and/or psychologically) upon repeated use and typically leads to abuse of the therapeutic agent. In this regard, the therapeutic agent can be any opioid agonist as discussed herein.

The therapeutic agent can be an opioid agonist. By “opioid” is meant to include a drug, hormone, or other chemical or biological substance, natural or synthetic, having a sedative, narcotic, or otherwise similar effect(s) to those containing opium or its natural or synthetic derivatives. By “opioid agonist,” sometimes used herein interchangeably with terms “opioid” and “opioid analgesic,” is meant to include one or more opioid agonists, either alone or in combination, and is further meant to include the base of the opioid, mixed or combined agonist-antagonists, partial agonists, pharmaceutically acceptable salts thereof, stereoisomers thereof, ethers thereof, esters thereof, and combinations thereof.

Opioid agonists include, for example, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenazocine, phenomorphan, phenoperidine, piminodine, piritramide, propheptazine, promedol, properidine, propiram, propoxyphene, sufentanil, tramadol, tilidine, derivatives or complexes thereof, pharmaceutically acceptable salts thereof, and combinations thereof. Preferably, the opioid agonist is selected from the group consisting of hydrocodone, hydromorphone, oxycodone, dihydrocodeine, codeine, dihydromorphine, morphine, buprenorphine, derivatives or complexes thereof, pharmaceutically acceptable salts thereof, and combinations thereof. Most preferably, the opioid agonist is morphine, hydromorphone, oxycodone or hydrocodone. In a preferred embodiment, the opioid agonist comprises oxycodone or hydrocodone and is present in the dosage form in an amount of about 15 to about 45 mg, and the opioid antagonist comprises naltrexone and is present in the dosage form in an amount of about 0.5 to about 5 mg.

Equianalgesic doses of these opioids, in comparison to a 15 mg dose of hydrocodone, are set forth in Table I below:

TABLE I
Equianalgesic Doses of Opioids

| Opioid | Calculated Dose (mg) |
|---|---|
| Oxycodone | 13.5 |
| Codeine | 90.0 |
| Hydrocodone | 15.0 |
| Hydromorphone | 3.375 |
| Levorphanol | 1.8 |
| Meperidine | 135.0 |
| Methadone | 9.0 |
| Morphine | 27.0 |

Hydrocodone is a semisynthetic narcotic analgesic and antitussive with multiple nervous system and gastrointestinal actions. Chemically, hydrocodone is 4,5-epoxy-3-methoxy-17-methylmorphinan-6-one, and is also known as dihydrocodeinone. Like other opioids, hydrocodone can be habit-forming and can produce drug dependence of the morphine type. Like other opium derivatives, excess doses of hydrocodone will depress respiration.

Oral hydrocodone is also available in Europe (e.g., Belgium, Germany, Greece, Italy, Luxembourg, Norway and Switzerland) as an antitussive agent. A parenteral formulation is also available in Germany as an antitussive agent. For use as an analgesic, hydrocodone bitartrate is commonly available in the United States only as a fixed combination with non-opiate drugs (e.g., ibuprofen, acetaminophen, aspirin; etc.) for relief of moderate to moderately severe pain.

A common dosage form of hydrocodone is in combination with acetaminophen and is commercially available, for example, as Lortab® in the United States from UCB Pharma, Inc. (Brussels, Belgium), as 2.5/500 mg, 5/500 mg, 7.5/500 mg and 10/500 mg hydrocodone/acetaminophen tablets. Tablets are also available in the ratio of 7.5 mg hydrocodone bitartrate and 650 mg acetaminophen and a 7.5 mg hydrocodone bitartrate and 750 mg acetaminophen. Hydrocodone, in combination with aspirin, is given in an oral dosage form to adults generally in 1-2 tablets every 4-6 hours as needed to alleviate pain. The tablet form is 5 mg hydrocodone bitartrate and 224 mg aspirin with 32 mg caffeine; or 5 mg hydrocodone bitartrate and 500 mg aspirin. Another formulation comprises hydrocodone bitartrate and ibuprofen. Vicoprofen®, commercially available in the U.S. from Knoll Laboratories (Mount Olive, N.J.), is a tablet containing 7.5 mg hydrocodone bitartrate and 200 mg ibuprofen. The invention is contemplated to encompass all such formulations, with the inclusion of the opioid antagonist and/or antagonist in sequestered form as part of a subunit comprising an opioid agonist.

Oxycodone, chemically known as 4,5-epoxy-14-hydroxy-3-methoxy-17-methylmorphinan-6-one, is an opioid agonist whose principal therapeutic action is analgesia. Other therapeutic effects of oxycodone include anxiolysis, euphoria and feelings of relaxation. The precise mechanism of its analgesic action is not known, but specific CNS opioid receptors for endogenous compounds with opioid-like activity have been identified throughout the brain and spinal cord and play a role in the analgesic effects of this drug.

Oxycodone is commercially available in the United States, e.g., as Oxycotin® from Purdue Pharma L.P. (Stamford, Conn.), as controlled-release tablets for oral administration containing 10 mg, 20 mg, 40 mg or 80 mg oxycodone hydrochloride, and as OxyIR™, also from Purdue Pharma as immediate-release capsules containing 5 mg oxycodone hydrochloride. The invention is contemplated to encompass all such formulations, with the inclusion of an opioid antagonist and/or antagonist in sequestered form as part of a subunit comprising an opioid agonist.

Oral hydromorphone is commercially available in the United States, e.g., as Dilaudid® from Abbott Laboratories (Chicago, Ill.). Oral morphine is commercially available in the United States, e.g., as Kadian® from Faulding Laboratories (Piscataway, N.J.).

Exemplary NSAIDS include ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam or isoxicam, and the like. Useful dosages of these drugs are well-known.

Exemplary NMDA receptor medicaments include morphinans, such as dexotromethorphan or dextrophan, ketamine, d-methadone, and pharmaceutically acceptable salts thereof, and encompass drugs that block a major intracellular consequence of NMDA-receptor activation, e.g., a ganglioside, such as (6-aminothexyl)-5-chloro-1-naphthalenesulfonamide. These drugs are stated to inhibit the development of tolerance to and/or dependence on addictive drugs, e.g., narcotic analgesics, such as morphine, codeine; etc., in U.S. Pat. Nos. 5,321,012 and 5,556,838 (both to Mayer et al.), both of which are incorporated herein by reference, and to treat chronic pain in U.S. Pat. No. 5,502,058 (Mayer et al.), incorporated herein by reference. The NMDA agonist can be included alone or in combination with a local anesthetic, such as lidocaine, as described in these patents by Mayer et al.

COX-2 inhibitors have been reported in the art, and many chemical compounds are known to produce inhibition of cyclooxygenase-2. COX-2 inhibitors are described, for example, in U.S. Pat. Nos. 5,616,601; 5,604,260; 5,593,994; 5,550,142; 5,536,752; 5,521,213; 5,475,995; 5,639,780; 5,604,253; 5,552,422; 5,510,368; 5,436,265; 5,409,944 and 5,130,311, all of which are incorporated herein by reference. Certain preferred COX-2 inhibitors include celecoxib (SC-58635), DUP-697, flosulide (CGP-28238), meloxicam, 6-methoxy-2-naphthylacetic acid (6-NMA), MK-966 (also known as Vioxx), nabumetone (prodrug for 6-MMA), nimesulide, NS-398, SC-5766, SC-58215, T-614, or combinations thereof. Dosage levels of COX-2 inhibitor on the order of from about 0.005 mg to about 140 mg per kilogram of body weight per day have been shown to be therapeutically effective in combination with an opioid analgesic. Alternatively, about 0.25 mg to about 7 g per patient per day of a COX-2 inhibitor can be administered in combination with an opioid analgesic.

The treatment of chronic pain via the use of glycine receptor antagonists and the identification of such drugs is described in U.S. Pat. No. 5,514,680 (Weber et al.), which is incorporated herein by reference.

Pharmaceutically acceptable salts of the antagonist or agonist agents discussed herein include metal salts, such as sodium salt, potassium salt, cesium salt, and the like; alkaline earth metals, such as calcium salt, magnesium salt, and the like; organic amine salts, such as triethylamine salt, pyridine salt, picoline salt, ethanolamine salt, triethanolamine salt, dicyclohexylamine salt, N,N′-dibenzylethylenediamine salt, and the like; inorganic acid salts, such as hydrochloride, hydrobromide, sulfate, phosphate, and the like; organic acid salts, such as formate, acetate, trifluoroacetate, maleate, tartrate, and the like; sulfonates, such as methanesulfonate, benzenesulfonate, p-toluenesulfonate, and the like; amino acid salts, such as arginate, asparginate, glutamate, and the like.

In embodiments in which the opioid agonist comprises hydrocodone, the sustained-release oral dosage forms can include analgesic doses from about 8 mg to about 50 mg of hydrocodone per dosage unit. In sustained-release oral dosage forms where hydromorphone is the therapeutically active opioid, it is included in an amount from about 2 mg to about 64 mg hydromorphone hydrochloride. In another embodiment, the opioid agonist comprises morphine, and the sustained-release oral dosage forms of the invention include from about 2.5 mg to about 800 mg morphine, by weight. In yet another embodiment, the opioid agonist comprises oxycodone and the sustained-release oral dosage forms include from about 2.5 mg to about 800 mg oxycodone. In certain preferred embodiments, the sustained-release oral dosage forms include from about 20 mg to about 30 mg oxycodone. Controlled release oxycodone formulations are known in the art. The following documents describe various controlled-release oxycodone formulations suitable for use in the invention described herein, and processes for their manufacture: U.S. Pat. Nos. 5,266,331; 5,549,912; 5,508,042; and 5,656,295, which are incorporated herein by reference. The opioid agonist can comprise tramadol and the sustained-release oral dosage forms can include from about 25 mg to 800 mg tramadol per dosage unit.

Methods of making any of the sequestering subunits of the invention are known in the art. See, for example, Remington: the Science and Practice of Pharmacy, Alfonso R. Genaro (ed), 20^{th} edition, and Example 2 set forth below. The sequestering subunits can be prepared by any suitable method to provide, for example, beads, pellets, granules, spheroids, and the like. Spheroids or beads, coated with an active ingredient can be prepared, for example, by dissolving the active ingredient in water and then spraying the solution onto a substrate, for example, nu pariel 18/20 beads, using a Wurster insert. Optionally, additional ingredients are also added prior to coating the beads in order to assist the active ingredient in binding to the substrates, and/or to color the solution; etc. The resulting substrate-active material optionally can be overcoated with a barrier material to separate the therapeutically active agent from the next coat of material, e.g., release-retarding material. Preferably, the barrier material is a material comprising hydroxypropyl methylcellulose. However, any film-former known in the art can be used. Preferably, the barrier material does not affect the dissolution rate of the final product.

Pellets comprising an active ingredient can be prepared, for example, by a melt pelletization technique. Typical of such techniques is when the active ingredient in finely divided form is combined with a binder (also in particulate form) and other optional inert ingredients, and thereafter the mixture is pelletized, e.g., by mechanically working the mixture in a high shear mixer to form the pellets (e.g., pellets, granules, spheres, beads; etc., collectively referred to herein as “pellets”). Thereafter, the pellets can be sieved in order to obtain pellets of the requisite size. The binder material is preferably in particulate form and has a melting point above about 40° C. Suitable binder substances include, for example, hydrogenated castor oil, hydrogenated vegetable oil, other hydrogenated fats, fatty alcohols, fatty acid esters, fatty acid glycerides, and the like.

The diameter of the extruder aperture or exit port also can be adjusted to vary the thickness of the extruded strands. Furthermore, the exit part of the extruder need not be round; it can be oblong, rectangular; etc. The exiting strands can be reduced to particles using a hot wire cutter, guillotine; etc.

The melt-extruded multiparticulate system can be, for example, in the form of granules, spheroids, pellets, or the like, depending upon the extruder exit orifice. The terms “melt-extruded multiparticulate(s)” and “melt-extruded multiparticulate system(s)” and “melt-extruded particles” are used interchangeably herein and include a plurality of subunits, preferably within a range of similar size and/or shape. The melt-extruded multiparticulates are preferably in a range of from about 0.1 to about 12 mm in length and have a diameter of from about 0.1 to about 5 mm. In addition, the melt-extruded multiparticulates can be any geometrical shape within this size range. Alternatively, the extrudate can simply be cut into desired lengths and divided into unit doses of the therapeutically active agent without the need of a spheronization step.

The substrate also can be prepared via a granulation technique. Generally, melt-granulation techniques involve melting a normally solid hydrophobic material, e.g., a wax, and incorporating an active ingredient therein. To obtain a sustained-release dosage form, it can be necessary to incorporate an additional hydrophobic material.

A coating composition can be applied onto a substrate by spraying it onto the substrate using any suitable spray equipment. For example, a Wurster fluidized-bed system can be used in which an air flow from underneath, fluidizes the coated material and effects drying, while the insoluble polymer coating is sprayed on. The thickness of the coating will depend on the characteristics of the particular coating composition, and can be determined by using routine experimentation.

Any manner of preparing a subunit can be employed. By way of example, a subunit in the form of a pellet or the like can be prepared by co-extruding a material comprising the opioid agonist and a material comprising the opioid antagonist and/or antagonist in sequestered form. Optionally, the opioid agonist composition can cover, e.g., overcoat, the material comprising the antagonist and/or antagonist in sequestered form. A bead, for example, can be prepared by coating a substrate comprising an opioid antagonist and/or an antagonist in sequestered form with a solution comprising an opioid agonist.

The sequestering subunits of the invention are particularly well-suited for use in compositions comprising the sequestering subunit and a therapeutic agent in releasable form. In this regard, the invention also provides a composition comprising any of the sequestering subunits of the invention and a therapeutic agent in releasable form. By “releasable form” is meant to include immediate release, intermediate release, and sustained-release forms. The therapeutic agent can be formulated to provide immediate release of the therapeutic agent. In preferred embodiments, the composition provides sustained-release of the therapeutic agent.

The therapeutic agent in sustained-release form is preferably a particle of therapeutic agent that is combined with a release-retarding material. The release-retarding material is preferably a material that permits release of the therapeutic agent at a sustained rate in an aqueous medium. The release-retarding material can be selectively chosen so as to achieve, in combination with the other stated properties, a desired in vitro release rate.

In a preferred embodiment, the oral dosage form of the invention can be formulated to provide for an increased duration of therapeutic action allowing once-daily dosing. In general, a release-retarding material is used to provide the increased duration of therapeutic action. Preferably, the once-daily dosing is provided by the dosage forms and methods described in U.S. Patent Application Pub. No. 2005/0020613 to Boehm, entitled “Sustained-Release Opioid Formulations and Method of Use,” filed on Sep. 22, 2003, and incorporated herein by reference.

Preferred release-retarding materials include acrylic polymers, alkylcelluloses, shellac, zein, hydrogenated vegetable oil, hydrogenated castor oil, and combinations thereof. In certain preferred embodiments, the release-retarding material is a pharmaceutically acceptable acrylic polymer, including acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, and glycidyl methacrylate copolymers. In certain preferred embodiments, the acrylic polymer comprises one or more ammonio methacrylate copolymers. Ammonio methacrylate copolymers are well-known in the art, and are, described in NF21, the 21^{st} edition of the National Formulary, published by the United States Pharmacopeial Convention Inc. (Rockville, Md.), as fully polymerized copolymers of acrylic and methacrylic acid esters with a low content of quaternary ammonium groups. In other preferred embodiments, the release-retarding material is an alkyl cellulosic material, such as ethylcellulose. Those skilled in the art will appreciate that other cellulosic polymers, including other alkyl cellulosic polymers, can be substituted for part or all of the ethylcellulose.

Release-modifying agents, which affect the release properties of the release-retarding material, also can be used. In a preferred embodiment, the release-modifying agent functions as a pore-former. The pore-former can be organic or inorganic, and include materials that can be dissolved, extracted or leached from the coating in the environment of use. The pore-former can comprise one or more hydrophilic polymers, such as hydroxypropylmethylcellulose. In certain preferred embodiments, the release-modifying agent is selected from hydroxypropylmethylcellulose, lactose, metal stearates, and combinations thereof.

The release-retarding material can also include an erosion-promoting agent, such as starch and gums; a release-modifying agent useful for making microporous lamina in the environment of use, such as polycarbonates comprised of linear polyesters of carbonic acid in which carbonate groups reoccur in the polymer chain; and/or a semi-permeable polymer.

The release-retarding material can also include an exit means comprising at least one passageway, orifice, or the like. The passageway can be formed by such methods as those disclosed in U.S. Pat. Nos. 3,845,770; 3,916,889; 4,063,064; and 4,088,864, which are incorporated herein by reference. The passageway can have any shape, such as round, triangular, square, elliptical, irregular; etc.

In certain embodiments, the therapeutic agent in sustained-release form can include a plurality of substrates comprising the active ingredient, which substrates are coated with a sustained-release coating comprising a release-retarding material.

The sustained-release preparations of the invention can be made in conjunction with any multiparticulate system, such as beads, ion-exchange resin beads, spheroids, microspheres, seeds, pellets, granules, and other multiparticulate systems in order to obtain a desired sustained-release of the therapeutic agent. The multiparticulate system can be presented in a capsule or in any other suitable unit dosage form.

In certain preferred embodiments, more than one multiparticulate system can be used, each exhibiting different characteristics, such as pH dependence of release, time for release in various media (e.g., acid, base, simulated intestinal fluid), release in vivo, size and composition.

To obtain a sustained-release of the therapeutic agent in a manner sufficient to provide a therapeutic effect for the sustained durations, the therapeutic agent can be coated with an amount of release-retarding material sufficient to obtain a weight gain level from about 2 to about 30%, although the coat can be greater or lesser depending upon the physical properties of the particular therapeutic agent utilized and the desired release rate, among other things. Moreover, there can be more than one release-retarding material used in the coat, as well as various other pharmaceutical excipients.

Solvents typically used for the release-retarding material include pharmaceutically acceptable solvents, such as water, methanol, ethanol, methylene chloride and combinations thereof.

In certain embodiments of the invention, the release-retarding material is in the form of a coating comprising an aqueous dispersion of a hydrophobic polymer. The inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticize the ethylcellulose before using the same as a coating material. Generally, the amount of plasticizer included in a coating solution is based on the concentration of the film-former, e.g., most often from about 1 to about 50 percent by weight of the film-former. Concentrations of the plasticizer, however, can be determined by routine experimentation.

Examples of plasticizers for ethylcellulose and other celluloses include dibutyl sebacate, diethyl phthalate, triethyl citrate, tributyl citrate, and triacetin, although it is possible that other plasticizers (such as acetylated monoglycerides, phthalate esters, castor oil; etc.) can be used.

Examples of plasticizers for the acrylic polymers include citric acid esters, such as triethyl citrate NF21, tributyl citrate, dibutyl phthalate, and possibly 1,2-propylene glycol, polyethylene glycols, propylene glycol, diethyl phthalate, castor oil, and triacetin, although it is possible that other plasticizers (such as acetylated monoglycerides, phthalate esters, castor oil; etc.) can be used.

The sustained-release profile of drug release in the formulations of the invention (either in vivo or in vitro) can be altered, for example, by using more than one release-retarding material, varying the thickness of the release-retarding material, changing the particular release-retarding material used, altering the relative amounts of release-retarding material, altering the manner in which the plasticizer is added (e.g., when the sustained-release coating is derived from an aqueous dispersion of hydrophobic polymer), by varying the amount of plasticizer relative to retardant material, by the inclusion of additional ingredients or excipients, by altering the method of manufacture; etc.

In certain other embodiments, the oral dosage form can utilize a multiparticulate sustained-release matrix. In certain embodiments, the sustained-release matrix comprises a hydrophilic and/or hydrophobic polymer, such as gums, cellulose ethers, acrylic resins and protein-derived materials. Of these polymers, the cellulose ethers, specifically hydroxyalkylcelluloses and carboxyalkylcelluloses, are preferred. The oral dosage form can contain between about 1% and about 80% (by weight) of at least one hydrophilic or hydrophobic polymer.

The hydrophobic material is preferably selected from the group consisting of alkylcellulose, acrylic and methacrylic acid polymers and copolymers, shellac, zein, hydrogenated castor oil, hydrogenated vegetable oil, or mixtures thereof. Preferably, the hydrophobic material is a pharmaceutically acceptable acrylic polymer, including acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylicacid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. In other embodiments, the hydrophobic material can also include hydrooxyalkylcelluloses such as hydroxypropylmethylcellulose and mixtures of the foregoing.

Preferred hydrophobic materials are water-insoluble with more or less pronounced hydrophobic trends. Preferably, the hydrophobic material has a melting point from about 30° C. to about 200° C., more preferably from about 45° C. to about 90° C. The hydrophobic material can include neutral or synthetic waxes, fatty alcohols (such as lauryl, myristyl, stearyl, cetyl or preferably cetostearyl alcohol), fatty acids, including fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones. Suitable waxes include beeswax, glycowax, castor wax, carnauba wax and wax-like substances, e.g., material normally solid at room temperature and having a melting point of from about 30° C. to about 100° C.

Preferably, a combination of two or more hydrophobic materials are included in the matrix formulations. If an additional hydrophobic material is included, it is preferably a natural or synthetic wax, a fatty acid, a fatty alcohol, or mixtures thereof. Examples include beeswax, carnauba wax, stearic acid and stearyl alcohol.

In other embodiments, the sustained-release matrix comprises digestible, long-chain (e.g., C₈-C₅₀, preferably C₁₂-C₄₀), substituted or unsubstituted hydrocarbons, such as fatty acids, fatty alcohols, glyceryl esters of fatty acids, mineral and vegetable oils and waxes. Hydrocarbons having a melting point of between about 25° C. and about 90° C. are preferred. Of these long-chain hydrocarbon materials, fatty (aliphatic) alcohols are preferred. The oral dosage form can contain up to about 60% (by weight) of at least one digestible, long-chain hydrocarbon.

Further, the sustained-release matrix can contain up to 60% (by weight) of at least one polyalkylene glycol.

In a preferred embodiment, the matrix comprises at least one water-soluble hydroxyalkyl cellulose, at least one C₁₂-C₃₆, preferably C₁₄-Cₙ, aliphatic alcohol and, optionally, at least one polyalkylene glycol. The at least one hydroxyalkyl cellulose is preferably a hydroxy (C₁-C₆) alkyl cellulose, such as hydroxypropylcellulose, hydroxypropylmethylcellulose and, preferably, hydroxyethyl cellulose. The amount of the at least one hydroxyalkyl cellulose in the oral dosage form will be determined, amongst other things, by the precise rate of opioid release required. The amount of the at least one aliphatic alcohol in the present oral dosage form will be determined by the precise rate of opioid release required. However, it will also depend on whether the at least one polyalkylene glycol is absent from the oral dosage form.

In certain embodiments, a spheronizing agent, together with the active ingredient, can be spheronized to form spheroids. Microcrystalline cellulose and hydrous lactose impalpable are examples of such agents. Additionally (or alternatively), the spheroids can contain a water-insoluble polymer, preferably an acrylic polymer, an acrylic copolymer, such as a methacrylic acid-ethyl acrylate copolymer, or ethyl cellulose. In such embodiments, the sustained-release coating will generally include a water-insoluble material such as (a) a wax, either alone or in admixture with a fatty alcohol, or (b) shellac or zein.

Preferably, the sequestering subunit comprises the therapeutic agent in sustained-release form. The sustained-release subunit can be prepared by any suitable method. For example, a plasticized aqueous dispersion of the release-retarding material can be applied onto the subunit comprising the opioid agonist. A sufficient amount of the aqueous dispersion of release-retarding material to obtain a predetermined sustained-release of the opioid agonist when the coated substrate is exposed to aqueous solutions, e.g., gastric fluid, is preferably applied, taking into account the physical characteristics of the opioid agonist, the manner of incorporation of the plasticizer; etc. Optionally, a further overcoat of a film-former, such as Opadry (Colorcon, West Point, Va.), can be applied after coating with the release-retarding material.

The subunit can be cured in order to obtain a stabilized release rate of the therapeutic agent. In embodiments employing an acrylic coating, a stabilized product can be preferably obtained by subjecting the subunit to oven curing at a temperature above the glass transition temperature of the plasticized acrylic polymer for the required time period. The optimum temperature and time for the particular formulation can be determined by routine experimentation.

Once prepared, the subunit can be combined with at least one additional subunit and, optionally, other excipients or drugs to provide an oral dosage form.

In addition to the above ingredients, a sustained-release matrix also can contain suitable quantities of other materials, e.g., diluents, lubricants, binders, granulating aids, colorants, flavorants and glidants that are conventional in the pharmaceutical art.

Optionally and preferably, the mechanical fragility of any of the sequestering subunits described herein is the same as the mechanical fragility of the therapeutic agent in releasable form. In this regard, tampering with the composition of the invention in a manner to obtain the therapeutic agent will result in the destruction of the sequestering subunit, such that the antagonist is released and mixed in with the therapeutic agent. Consequently, the antagonist cannot be separated from the therapeutic agent, and the therapeutic agent cannot be administered in the absence of the antagonist. Methods of assaying the mechanical fragility of the sequestering subunit and of a therapeutic agent are known in the art.

The composition of the invention can be in any suitable dosage form or formulation, (see, e.g., Pharmaceutics and Pharmacy Practice, J. B. Lippincott Company, Philadelphia, Pa., Banker and Chalmers, eds., pages 238-250 (1982)). Formulations suitable for oral administration can consist of (a) liquid solutions, such as an effective amount of the inhibitor dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

One of ordinary skill in the art will readily appreciate that the compositions of the invention can be modified in any number of ways, such that the therapeutic efficacy of the composition is increased through the modification. For instance, the therapeutic agent or sequestering subunit could be conjugated either directly or indirectly through a linker to a targeting moiety. The practice of conjugating therapeutic agents or sequestering subunits to targeting moieties is known in the art. See, for instance, Wadwa et al., J. Drug Targeting 3: 111 (1995), and U.S. Pat. No. 5,087,616. The term “targeting moiety” as used herein, refers to any molecule or agent that specifically recognizes and binds to a cell-surface receptor, such that the targeting moiety directs the delivery of the therapeutic agent or sequestering subunit to a population of cells on which the receptor is expressed. Targeting moieties include, but are not limited to, antibodies, or fragments thereof, peptides, hormones, growth factors, cytokines, and any other naturally- or non-naturally-existing ligands, which bind to cell-surface receptors. The term “linker” as used herein, refers to any agent or molecule that bridges the therapeutic agent or sequestering subunit to the targeting moiety. One of ordinary skill in the art recognizes that sites on the therapeutic agent or sequestering subunit, which are not necessary for the function of the agent or sequestering subunit, are ideal sites for attaching a linker and/or a targeting moiety, provided that the linker and/or targeting moiety, once attached to the agent or sequestering subunit, do(es) not interfere with the function of the therapeutic agent or sequestering subunit.

With respect to the present inventive compositions, the composition is preferably an oral dosage form. By “oral dosage form” is meant to include a unit dosage form prescribed or intended for oral administration comprising subunits. Desirably, the composition comprises the sequestering subunit coated with the therapeutic agent in releasable form, thereby forming a composite subunit comprising the sequestering subunit and the therapeutic agent. Accordingly, the invention further provides a capsule suitable for oral administration comprising a plurality of such composite subunits.

Alternatively, the oral dosage form can comprise any of the sequestering subunits of the invention in combination with a therapeutic agent subunit, wherein the therapeutic agent subunit comprises the therapeutic agent in releasable form. In this respect, the invention provides a capsule suitable for oral administration comprising a plurality of sequestering subunits of the invention and a plurality of therapeutic subunits, each of which comprises a therapeutic agent in releasable form.

The invention further provides tablets comprising a sequestering subunit of the invention and a therapeutic agent in releasable form. For instance, the invention provides a tablet suitable for oral administration comprising a first layer comprising any of the sequestering subunits of the invention and a second layer comprising therapeutic agent in releasable form, wherein the first layer is coated with the second layer. The first layer can comprise a plurality of sequestering subunits. Alternatively, the first layer can be or can consist of a single sequestering subunit. The therapeutic agent in releasable form can be in the form of a therapeutic agent subunit and the second layer can comprise a plurality of therapeutic subunits. Alternatively, the second layer can comprise a single substantially homogeneous layer comprising the therapeutic agent in releasable form.

When the blocking agent is a system comprising a first antagonist-impermeable material and a core, the sequestering subunit can be in one of several different forms. For example, the system can further comprise a second antagonist-impermeable material, in which case the sequestering unit comprises an antagonist, a first antagonist-impermeable material, a second antagonist-impermeable material, and a core. In this instance, the core is coated with the first antagonist-impermeable material, which, in turn, is coated with the antagonist, which, in turn, is coated with the second antagonist-impermeable material. The first antagonist-impermeable material and second antagonist-impermeable material substantially prevent release of the antagonist from the sequestering subunit in the gastrointestinal tract for a time period that is greater than 24 hours. In some instances, it is preferable that the first antagonist-impermeable material is the same as the second antagonist-impermeable material. In other instances, the first antagonist-impermeable material is different from the second antagonist-impermeable material. It is within the skill of the ordinary artisan to determine whether or not the first and second antagonist-impermeable materials should be the same or different. Factors that influence the decision as to whether the first and second antagonist-impermeable materials should be the same or different can include whether a layer to be placed over the antagonist-impermeable material requires certain properties to prevent dissolving part or all of the antagonist-impermeable layer when applying the next layer or properties to promote adhesion of a layer to be applied over the antagonist-impermeable layer.

Alternatively, the antagonist can be incorporated into the core, and the core is coated with the first antagonist-impermeable material. In this case, the invention provides a sequestering subunit comprising an antagonist, a core and a first antagonist-impermeable material, wherein the antagonist is incorporated into the core and the core is coated with the first antagonist-impermeable material, and wherein the first antagonist-impermeable material substantially prevents release of the antagonist from the sequestering subunit in the gastrointestinal tract for a time period that is greater than 24 hours. By “incorporate” and words stemming therefrom, as used herein is meant to include any means of incorporation, e.g., homogeneous dispersion of the antagonist throughout the core, a single layer of the antagonist coated on top of a core, or a multi-layer system of the antagonist, which comprises the core.

In another alternative embodiment, the core comprises a water-insoluble material, and the core is coated with the antagonist, which, in turn, is coated with the first antagonist-impermeable material. In this case, the invention further provides a sequestering subunit comprising an antagonist, a first antagonist-impermeable material, and a core, which comprises a water-insoluble material, wherein the core is coated with the antagonist, which, in turn, is coated with the first antagonist-impermeable material, and wherein the first antagonist-impermeable material substantially prevents release of the antagonist from the sequestering subunit in the gastrointestinal tract for a time period that is greater than 24 hours. The term “water-insoluble material” as used herein means any material that is substantially water-insoluble. The term “substantially water-insoluble” does not necessarily refer to complete or 100% water-insolubility. Rather, there are varying degrees of water insolubility of which one of ordinary skill in the art recognizes as having a potential benefit. Preferred water-insoluble materials include, for example, microcrystalline cellulose, a calcium salt, and a wax. Calcium salts include, but are not limited to, a calcium phosphate (e.g., hydroxyapatite, apatite; etc.), calcium carbonate, calcium sulfate, calcium stearate, and the like. Waxes include, for example, carnuba wax, beeswax, petroleum wax, candelilla wax, and the like.

In one embodiment, the sequestering subunit includes an antagonist and a seal coat where the seal coat forms a layer physically separating the antagonist within the sequestering subunit from the agonist which is layered upon the sequestering subunit. In one embodiment, the seal coat comprises one or more of an osmotic pressure regulating agent, a charge-neutralizing additive, a sequestering polymer hydrophobicity-enhancing additive, and a first sequestering polymer (each having been described above). In such embodiments, it is preferred that the osmotic pressure regulating agent, charge-neutralizing additive, and/or sequestering polymer hydrophobicity-enhancing additive, respectively where present, are present in proportion to the first sequestering polymer such that no more than 10% of the antagonist is released from the intact dosage form. Where an opioid antagonist is used in the sequestering subunit and the intact dosage form includes an opioid agonist, it is preferred that ratio of the osmotic pressure regulating agent, charge-neutralizing additive, and/or sequestering polymer hydrophobicity-enhancing additive, respectively where present, in relation to the first sequestering polymer is such that the physiological effect of the opioid agonist is not diminished when the composition is in its intact dosage form or during the normal course digestion in the patient. Release may be determined as described above using the USP paddle method (optionally using a buffer containing a surfactant such as Triton X-100) or measured from plasma after administration to a patient in the fed or non-fed state. In one embodiment, plasma naltrexone levels are determined; in others, plasma 6-beta naltrexol levels are determined. Standard tests may be utilized to ascertain the antagonist's effect on agonist function (i.e., reduction of pain).

The sequestering subunit of the invention can have a blocking agent that is a tether to which the antagonist is attached. The term “tether” as used herein refers to any means by which the antagonist is tethered or attached to the interior of the sequestering subunit, such that the antagonist is not released, unless the sequestering subunit is tampered with. In this instance, a tether-antagonist complex is formed. The complex is coated with a tether-impermeable material, thereby substantially preventing release of the antagonist from the subunit. The term “tether-impermeable material” as used herein refers to any material that substantially prevents or prevents the tether from permeating through the material. The tether preferably is an ion exchange resin bead.

The invention further provides a tablet suitable for oral administration comprising a single layer comprising a therapeutic agent in releasable form and a plurality of any of the sequestering subunits of the invention dispersed throughout the layer of the therapeutic agent in releasable form. The invention also provides a tablet in which the therapeutic agent in releasable form is in the form of a therapeutic agent subunit and the tablet comprises an at least substantially homogeneous mixture of a plurality of sequestering subunits and a plurality of subunits comprising the therapeutic agent.

In preferred embodiments, oral dosage forms are prepared to include an effective amount of melt-extruded subunits in the form of multiparticles within a capsule. For example, a plurality of the melt-extruded muliparticulates can be placed in a gelatin capsule in an amount sufficient to provide an effective release dose when ingested and contacted by gastric fluid. In another preferred embodiment, the subunits, e.g., in the form of multiparticulates, can be compressed into an oral tablet using conventional tableting equipment using standard techniques. Techniques and compositions for making tablets (compressed and molded), capsules (hard and soft gelatin) and pills are also described in Remingion's Pharmaceutical Sciences, (Aurther Osol., editor), 1553-1593 (1980), which is incorporated herein by reference. Excipients in tablet formulation can include, for example, an inert diluent such as lactose, granulating and disintegrating agents, such as cornstarch, binding agents, such as starch, and lubricating agents, such as magnesium stearate.

In yet another preferred embodiment, the subunits are added during the extrusion process and the extrudate can be shaped into tablets as set forth in U.S. Pat. No. 4,957,681 (Klimesch et al), which is incorporated herein by reference.

Optionally, the sustained-release, melt-extruded, multiparticulate systems or tablets can be coated, or the gelatin capsule can be further coated, with a sustained-release coating, such as the sustained-release coatings described herein. Such coatings are particularly useful when the subunit comprises an opioid agonist in releasable form, but not in sustained-release form. The coatings preferably include a sufficient amount of a hydrophobic material to obtain a weight gain level form about 2 to about 30 percent, although the overcoat can be greater, depending upon the physical properties of the particular opioid analgesic utilized and the desired release rate, among other things.

The melt-extruded dosage forms can further include combinations of melt-extruded multiparticulates containing one or more of the therapeutically active agents before being encapsulated. Furthermore, the dosage forms can also include an amount of an immediate release therapeutic agent for prompt therapeutic effect. The immediate release therapeutic agent can be incorporated or coated on the surface of the subunits after preparation of the dosage forms (e.g., controlled-release coating or matrix-based). The dosage forms can also contain a combination of controlled-release beads and matrix multiparticulates to achieve a desired effect.

The sustained-release formulations preferably slowly release the therapeutic agent, e.g., when ingested and exposed to gastric fluids, and then to intestinal fluids. The sustained-release profile of the melt-extruded formulations can be altered, for example, by varying the amount of retardant, e.g., hydrophobic material, by varying the amount of plasticizer relative to hydrophobic material, by the inclusion of additional ingredients or excipients, by altering the method of manufacture; etc.

In other embodiments, the melt-extruded material is prepared without the inclusion of the subunits, which are added thereafter to the extrudate. Such formulations can have the subunits and other drugs blended together with the extruded matrix material, and then the mixture is tableted in order to provide a slow release of the therapeutic agent or other drugs. Such formulations can be particularly advantageous, for example, when the therapeutically active agent included in the formulation is sensitive to temperatures needed for softening the hydrophobic material and/or the retardant material.

In certain embodiments, the release of the antagonist of the sequestering subunit or composition is expressed in terms of a ratio of the release achieved after tampering, e.g., by crushing or chewing, relative to the amount released from the intact formulation. The ratio is, therefore, expressed as [Crushed]:[Whole], and it is desired that this ratio have a numerical range of at least about 4:1 or greater (e.g., crushed release within 1 hour/intact release in 24 hours). In certain embodiments, the ratio of the therapeutic agent and the antagonist, present in the sequestering subunit, is about 1:1, about 50:1, about 75:1, about 100:1, about 150:1, or about 200:1, for example, by weight, preferably about 1:1 to about 20:1 by weight or 15:1 to about 30:1 by weight. The weight ratio of the therapeutic agent to antagonist refers to the weight of the active ingredients. Thus, for example, the weight of the therapeutic agent excludes the weight of the coating, matrix, or other component that renders the antagonist sequestered, or other possible excipients associated with the antagonist particles. In certain preferred embodiments, the ratio is about 1:1 to about 10:1 by weight. Because in certain embodiments the antagonist is in a sequestered from, the amount of such antagonist within the dosage form can be varied more widely than the therapeutic agent/antagonist combination dosage forms, where both are available for release upon administration, as the formulation does not depend on differential metabolism or hepatic clearance for proper functioning. For safety reasons, the amount of the antagonist present in a substantially non-releasable form is selected as not to be harmful to humans, even if fully released under conditions of tampering.

The compositions of the invention are particularly well-suited for use in preventing abuse of a therapeutic agent. In this regard, the invention also provides a method of preventing abuse of a therapeutic agent by a human being. The method comprises incorporating the therapeutic agent into any of the compositions of the invention. Upon administration of the composition of the invention to the person, the antagonist is substantially prevented from being released in the gastrointestinal tract for a time period that is greater than 24 hours. However, if a person tampers with the compositions, the sequestering subunit, which is mechanically fragile, will break and thereby allow the antagonist to be released. Since the mechanical fragility of the sequestering subunit is the same as the therapeutic agent in releasable form, the antagonist will be mixed with the therapeutic agent, such that separation between the two components is virtually impossible.

The effectiveness of treatment of chronic moderate to severe pain (focusing on osteoarthritis of the hip or knee) is typically measured by mean change in diary Brief Pain Inventory (BPI) score of average pain (daily scores of average pain averaged over 7 days; in-clinic BPI and/or daily diary BPI (worst, least, and current pain)), WOMAC Osteoarthritis Index, Medical Outcomes Study (MOS) Sleep Scale, Beck Depression Inventory, and Patient Global Impression of Change (PGIC). The safety and tolerability of opioid medications such as Kadian NT are compared to placebo using Adverse Events (AEs), clinical laboratory data, vital signs, and two measures of opioid withdrawal: Subjective Opiate Withdrawal Scale (SOWS) and Clinical Opiate Withdrawal Scale (COWS).

BPI is typically measured using 11-point BPI system as follows:
1. Please rate your pain by circling the one number that best describes your pain at its worst in the last 24 hours.

| | | | | | | | | | | |
|---|---|---|---|---|---|---|---|---|---|---|
| 0 | 1 | 2 | 3 | 4 | 5 | 6 | 7 | 8 | 9 | 10 |

| | |
|---|---|
| No pain | Pain as bad as |
| | you can imagine |

2. Please rate your pain by circling the one number that best describes your pain at its least in the last 24 hours.

| | | | | | | | | | | |
|---|---|---|---|---|---|---|---|---|---|---|
| 0 | 1 | 2 | 3 | 4 | 5 | 6 | 7 | 8 | 9 | 10 |

| | |
|---|---|
| No pain | Pain as bad as |
| | you can imagine |

3. Please rate your pain by circling the one number that best describes your pain on the average in the last 24 hours.

| | | | | | | | | | | |
|---|---|---|---|---|---|---|---|---|---|---|
| 0 | 1 | 2 | 3 | 4 | 5 | 6 | 7 | 8 | 9 | 10 |

| | |
|---|---|
| No pain | Pain as bad as |
| | you can imagine |

4. Please rate your pain by circling the one number that tells how much pain you have right now.

| | | | | | | | | | | |
|---|---|---|---|---|---|---|---|---|---|---|
| 0 | 1 | 2 | 3 | 4 | 5 | 6 | 7 | 8 | 9 | 10 |

| | |
|---|---|
| No pain | Pain as bad as |
| | you can imagine |

The MOS Sleep Scale is a self-administered, subject-rated questionnaire consisting of 12 items that assess key components of sleep (R. D., & Stewart, A. L. (1992). Sleep measures. In A. L. Stewart & J. E. Ware (eds.), Measuring functioning and well-being: The Medical Outcomes Study approach (pp. 235-259), Durham, N.C.: Duke University Press). When scored, the instrument provides seven subscale scores (sleep disturbance, snoring, awaken short of breath or with a headache, quantity of sleep, optimal sleep, sleep adequacy, and somnolence) as well as a nine-item overall sleep problems index. Higher scores reflect more impairment in all subscales except for sleep adequacy, where a higher score reflects less impairment. A typical representation of the MOS Sleep Scale is shown below:

1. How long did it usually take for you to fall asleep during the past four weeks?

| | (Circle One) |
|---|---|
| 0-15 minutes | 1 |
| 16-30 minutes | 2 |
| 31-45 minutes | 3 |
| 46-60 minutes | 4 |
| More than 60 minutes | 5 |

2. On the average, how many hours did you sleep each night during the past four weeks?

| | |
|---|---|
| Write in the number of | □□ |
| hours per night: | |

How often during the past four weeks did you . . . .

| | | (Circle One Number On Each Line) | | | | | |
|---|---|---|---|---|---|---|---|
| | | | | A Good | | | |
| | | All of | Most | Bit of | Some | A Little | None |
| | | the | of the | the | of the | of the | of the |
| | | Time | Time | Time | Time | Time | Time |
| | | ▾ | ▾ | ▾ | ▾ | ▾ | ▾ |
| 3. | feel that your sleep was not | 1 | 2 | 3 | 4 | 5 | 6 |
| | quiet (moving restlessly, | | | | | | |
| | feeling tense, speaking, etc., | | | | | | |
| | while sleeping)? | | | | | | |
| 4. | get enough sleep to feel rested | 1 | 2 | 3 | 4 | 5 | 6 |
| | upon waking in the morning? | | | | | | |
| 5. | awaken short of breath or with | 1 | 2 | 3 | 4 | 5 | 6 |
| | a headache? | | | | | | |
| 6. | feel drowsy or sleepy during | 1 | 2 | 3 | 4 | 5 | 6 |
| | the day? | | | | | | |
| 7. | have trouble falling asleep? | 1 | 2 | 3 | 4 | 5 | 6 |
| 8. | awaken during your sleep | 1 | 2 | 3 | 4 | 5 | 6 |
| | time and have trouble falling | | | | | | |
| | asleep again? | | | | | | |
| 9. | have trouble staying awake | 1 | 2 | 3 | 4 | 5 | 6 |
| | during the day? | | | | | | |
| 10. | snore during your sleep? | 1 | 2 | 3 | 4 | 5 | 6 |
| 11. | take naps (5 minutes or | 1 | 2 | 3 | 4 | 5 | 6 |
| | longer) during the day? | | | | | | |
| 12. | get the amount of sleep you | 1 | 2 | 3 | 4 | 5 | 6 |
| | needed? | | | | | | |

The Beck Depression Inventory is a self-administered, 21-item test in multiple-choice format that measures the presence and degree of depression (Beck et al. An inventory for measuring depression. Arch Gen Psych. 1961; 4:561-571). Each of the inventory questions corresponds to a specific category of depressive symptom and/or attitude. Answers are scored on a 0 to 3 scale, where “0” is minimal and “3” is severe. A score of <15 indicates mild depression, a score of 15-30 indicates moderate depression, and a score >30 indicates severe depression.

The WOMAC Osteoarthritis Index consists of questions on three subscales: Pain, Stiffness, and Physical Function (Bellamy et al. Validation study of WOMAC: a health status instrument for measuring clinically important patient relevant outcomes to antirheumatic drug therapy in patients with osteoarthritis of the hip or knee. J Rheumatol. 1988; 15:1833-1840; Bellamy N. Pain assessment in osteoarthritis: experience with the WOMAC osteoarthritis index. Semin Arthritis Rheum. 1989; 18:14-17; Bellamy et al. Double blind randomized controlled trial of sodium meclofenamate (Meclomen) and diclofenac sodium (Voltaren): post validation reapplication of the WOMAC Osteoarthritis index. J Rheumatol. 1992; 19:153-159). Questions are typically completed by the subject before any other efficacy assessments are performed. A typical WOMAC survey is reproduced below:

The PGIC is a self-administered instrument that measures change in patient's overall status on a scale ranging from 1 (very much improved) to 7 (very much worse). The PGIC is based on the Clinical Global Impression of Change (CGIC) (Guy W. ECDEU assessment manual for psychopharmacology. Washington, D.C.: Department of Health, Education and Welfare, 1976; 217-222. Publication Number (ADM) 76-338), which is a validated scale. A typical form of the PGIC survey is shown below:
How would you rate your overall status since your last visit?

(Please circle one)

| | |
|---|---|
| Very Much Improved | 1 |
| Much Improved | 2 |
| Minimally Improved | 3 |
| No Change | 4 |
| Minimally Worse | 5 |
| Much Worse | 6 |
| Very Much Worse | 7 |

Any or all of these measures of effectiveness may be used alone or in combination to determine the efficacy of various formulations or treatment regimens. Provided herein are methods for treating pain in a person comprising administering thereto a multilayer pharmaceutical composition as described herein such that pain is substantially relieved in the patient. By “substantially relieved” is meant that the person reports a decrease in pain as measured by any of several known methods (including but not limited to those described herein) for determining pain. This decrease may be in comparison to no treatment, a placebo, or another form of treatment including but not limited to another composition, either one described herein or otherwise available to one of skill in the art. Typically but not necessarily, pain is considered substantially relieved where the decrease is significant (e.g., p<0.05). The methods described herein provide methods for substantially relieving pain (e.g, providing an analgesic effect) for time periods of at least one week (e.g., two, four, eight, 12, 16, 20, 24, 28, 32, 36, 40 and 100 weeks) by administering a multi-layer pharmaceutical composition as described herein. In one embodiment, the method includes regularly administering (e.g., at least once, twice, three, or four times daily) a multi-layer pharmaceutical composition comprising an agonist and an atagonist as described herein for at least one week (e.g., one, two, four, eight, 12, 16, 20, 24, 28, 32, 36, 40 and 100 weeks) wherein no substantial release (e.g., zero, or less than about 10%, 20%, or 30% release) of the antagonist is observed. In some embodiments, administration of the composition to a population once daily for a time period of at least one week results in no substantial release in at least about 90%, 80%, 70%, 60%, or 50% of the individuals making up the population. Release may be measured by detecting naltrexone or β-naltrexol in plasma.

A better understanding of the present invention and of its many advantages will be had from the following examples, given by way of illustration.

### EXAMPLES

The preparations and experiments described below were actually performed. In certain cases, however, the present tense is utilized.

Exemplary KadianNT formulations and methods described below in Examples 1-4 may also be found in PCT/US2007/014282 (WO 2007/149438 A2), PCT/US2007/021627 (WO 2008/063301 A2), and PCT/US08/10357.

### Example 1

### Optimization Study #4, KadianNT, Morphine Sulfate and Naltreronc 60 mg/4.8 mg

### (20-780-1N)

| | PI-1495 | | PI-1496 | |
|---|---|---|---|---|
| | mg/unit | Percent | mg/unit | Percent |
| Sealed-coated sugar spheres | | | | |
| Sugar spheres (#25-30 mesh) | 37.2 | 11.7 | 37.1 | 11.9 |
| Ethylcellulose N50 | 6.2 | 1.9 | 6.2 | 2.0 |
| Mag Stearate | 2.5 | 0.8 | 2.5 | 0.8 |
| DBS | 0.6 | 0.2 | 0.6 | 0.2 |
| Talc | 15.5 | 4.9 | 15.5 | 5.0 |
| Subtotal | 62.0 | 19.4 | 61.9 | 19.9 |
| Naltrexone cores | | | | |
| Sealed sugar spheres | (62.0) | (19.4) | (61.9) | (19.9) |
| Naltrexone HCl | 4.8 | 1.50 | 4.8 | 1.54 |
| HPC (Klucel LF) | 0.9 | 0.3 | 0.9 | 0.3 |
| Ascorbic acid | 0.5 | 0.2 | 0.5 | 0.2 |
| Talc | 2.27 | 0.7 | 2.24 | 0.7 |
| Subtotal | 70.5 | 22.1 | 70.3 | 22.6 |
| Naltrexone pellets | | | | |
| Naltrezone cores | (70.5) | (22.1) | (70.3) | (22.6) |
| Eudragit RS PO | 53.3 | 16.7 | 53.3 | 17.1 |
| SLS | 1.8 | 0.6 | 1.8 | 0.6 |
| DBS | 5.36 | 1.7 | 5.36 | 1.7 |
| Talc | 52.1 | 16.3 | 52.1 | 16.8 |
| Subtotal | 183.0 | 57.4 | 182.9 | 58.8 |
| Naltrexone-morphine cores | | | | |
| Naltrexone pellets | (183.0) | (57.4) | (182.9) | (58.8) |
| Morphine sulfate | 59.9 | 18.8 | 59.7 | 19.2 |
| Sodium chloride | 11.2 | 3.5 | | |
| HPC (Klucel LF) | 7.3 | 2.3 | 4.76 | 1.5 |
| HPMC, 3 cps | | | 7.6 | 2.4 |
| Subtotal | 261.4 | 82.0 | 255.0 | 82.0 |
| Naltrexone-morphine pellets | | | | |
| Naltrexone-morphine cores | (261.4) | (82.0) | (255.0) | (82.0) |
| Ethylcellulose N50 | 19.81 | 6.2 | 19.31 | 6.2 |
| PEG 6000 | 9.16 | 2.9 | 8.9 | 2.9 |
| Eudragit L100-55 | 4.3 | 1.3 | 4.2 | 1.4 |
| DEP | 4.12 | 1.3 | 4 | 1.3 |
| Talc | 20.13 | 6.3 | 19.62 | 6.3 |
| Total | 319.0 | 100.0 | 311.0 | 100.0 |

### A. Method of Preparation—

1. Dissolve Ethylcellulose and dibutyl sebacate into ethanol, then disperse talc and magnesium stearate into the solution.
2. Spray the dispersion from 1 onto sugar spheres in a Wurster to form seal-coated sugar spheres (50 μm seal coat).
3. Dissolve Klucel LF and ascorbic acid into 20:80 mixture of water and ethanol. Disperse naltrexone HCl and talc into the solution.
4. Spray the naltrexone dispersion from 3 onto seal-coated sugar spheres from 2 in a Wurster to form naltrexone cores.
5. Dissolve Eudragit RS, sodium lauryl sulfate and dibutyl debacate into ethanol. Disperse talc into the solution.
6. Spray the dispersion from 5 onto naltrexone cores from 4 in a Wurster to form naltrexone pellets.
7. The Naltrexone pellets are dried at 50° C. for 48 hours.
8. Resulting pellets have a Eudragit RS coat thickness of 150 μm for both PI-1495 PI-1496.
9. (Only for PI-1495) Dissolve sodium chloride and hypromellose into water.
10. Dissolve hypromellose into 10:90 mixture of water and ethanol. Disperse morphine sulfate into the solution.
11. (Only for PI-1495) Spray the solution from 9 followed by the dispersion from 10 onto naltrexone pellets in 7 in a rotor to form naltrexone-morphine cores.
12. (Only for PI-1496) Spray the dispersion from 10 onto naltrexone pellets in 7 in a rotor to form naltrexone-morphine cores.
13. Dissolve ethylcellulose, PEG 6000, Eudragit L100-55 and diethyl phthalate into ethanol. Disperse talc into the solution.
14. Spray the dispersion from 12 onto naltrexone-morphine cores in 11 or 12 to form naltrexone-morphine pellets.
15. The pellets are filled into capsules.

### B. In-Vitro Drug Release—

1. Method—USP paddle method at 37° C. and 100 rpm
   1 hour in 0.1N HCl, then 72 hours in 0.05M pH 7.5 phosphate buffer
Results—Percent of NT released at 73 hours for PI-1495=0%
   Percent of NT released at 73 hours for PI-1496=0%
2. Method—USP paddle method at 37° C. and 100 rpm
   72 hrs in 0.2% Triton X-100/0.2% sodium acetate/0.002N HCl, pH 5.5
Results—Percent of NT released at 73 hours for PI-1495=0%
   Percent of NT released at 73 hours for PI-1496=0%

### C. In-Vivo Study

This is a single-dose, open-label, two period study in which two groups of eight subjects received one dose of either PI-1495 or PI-1496. Each subject received an assigned treatment sequence based on a randomization schedule under fasting and non-fasting conditions. Blood samples were drawn prior to dose administration and at 0.5 to 168 hours post-dose. Limits of quantitation are 4.00 pg/mL for naltrexone and 0.250 pg/mL for 6-beta-naltrexol. A summary of the pharmacokinetic results is shown in the following tables.

Naltrexone

| | PI-1495 | | PI-1496 | |
|---|---|---|---|---|
| | Fast | Fed | Fast | Fed |
| Tmax (hr) | 54.00 (N = 2) | 14.34 (N = 3) | 55.20 (N = 5) | 41.60 (N = 5) |
| Cmax (pg/mL) | 8.53 | 6.32 (N = 7) | 24.23 (N = 7) | 45.67 (N = 7) |
| AUCₗₐₛₜ (pg*h/mL) | 100.8 | 75.9 (N = 7) | 500.6 (N = 7) | 1265 (N = 7) |
| AUC∞ (pg*h/mL) | — | — | 2105.3 (N = 2) | 3737 (N = 2) |
| T½ (hr) | — | — | 44.56 (N = 2) | 33.17 (N = 2) |

Relative Bioavailability to an oral solution (Dose-adjusted)

| | | | | |
|---|---|---|---|---|
| Cmax Ratio (Test/Solution) | 0.29% | 0.21% | 0.82% | 1.55% |
| AUCₗₐₛₜ Ratio (Test/Solution) | 1.13% | 0.85% | 5.61% | 14.17% |
| AUC∞ Ratio (Test/Solution) | — | — | 22.0% | 39.1% |

N = 8, unless specified otherwise

6-beta-Naltrexol

| | PI-1495 | | PI-1496 | |
|---|---|---|---|---|
| | Fast | Fed | Fast | Fed |
| Tmax (hr) | 69.00 | 41.44 (N = 7) | 70.51 | 67.63 |
| Cmax (pg/mL) | 116.3 | 151.7 (N = 7) | 303.3 | 656.7 |
| AUCₗₐₛₜ | 5043 | 7332 (N = 7) | 14653 | 27503 |
| (pg*h/mL) | | | | |
| AUC∞ | 5607 | 8449 (N = 6) | 14930 | 27827 |
| (pg*h/mL) | | | | |
| T½ (hr) | 20.97 | 16.69 (N = 7) | 16.29 | 22.59 |

Relative Bioavailability to an oral solution (Dose-adjusted)

| | | | | |
|---|---|---|---|---|
| Cmax Ratio | 0.47% | 0.62% | 1.23% | 2.67% |
| (Test/Solution) | | | | |
| AUCₗₐₛₜ Ratio | 2.45% | 3.45% | 7.12% | 13.36% |
| (Test/Solution) | | | | |
| AUC∞ Ratio | 2.64% | 3.97% | 7.02% | 13.08% |
| (Test/Solution) | | | | |

N = 8, unless specified otherwise

Kadian NT pellets with naltrexone pellet coat thickness of 150 μm had comparable naltrexone release as NT pellets with 90 μm coat thickness. This comparable NT release may also be attributed from the presence of 50 μm seal coat on the sugar spheres used in Kadian NT pellets. Significant NT sequestering was observed, both at fasting (>97%) and fed states (>96%). Kadian NT pellets containing sodium chloride immediately above the naltrexone pellet coat (PI-1495) had half the release of naltrexone compared to Kadian NT pellet without sodium chloride (PI-1496), consistent with in vitro results. There is again food effect observed. Lag time was significantly reduced.

### Example 2

### Optimization Study #5, KadianNT, Morphine Sulfate and Naltrexone HCl 60 mg/2.4 mg (20-963-AU)

PI-1510

| | Mg/unit | Percent |
|---|---|---|
| Sealed sugar spheres | | |
| Sugar spheres (#25-30 mesh) | 39.9 | 12.2 |
| Ethylcellulose N50 | 6.5 | 2.0 |
| Mag Stearate | 2.6 | 0.8 |
| DBS | 0.7 | 0.2 |
| Talc | 16.7 | 5.1 |
| Subtotal | 66.4 | 20.3 |
| Naltrexone cores | | |
| Sealed sugar spheres | (66.4) | (20.3) |
| Naltrexone HCl | 2.4 | 0.73 |
| HPC (Klucel LF) | 0.5 | 0.1 |
| Ascorbic acid | 0.2 | 0.1 |
| Talc | 1.1 | 0.4 |
| Subtotal | 70.6 | 21.6 |
| Naltrexone pellets | | |
| Naltrexone cores | (70.6) | (21.6) |
| Eudragit RS PO | 53.0 | 16.2 |
| SLS | 1.8 | 0.6 |
| DBS | 5.3 | 1.6 |
| Talc | 53.0 | 16.2 |
| Subtotal | 183.7 | 56.2 |
| Naltrexone-morphine cores | | |
| Naltrexone pellets | (183.7) | (56.2) |
| Morphine sulfate | 60.1 | 18.4 |
| Sodium chloride | 12.5 | 3.8 |
| HPC (Klucel LF) | 6.2 | 1.9 |
| Subtotal | 262.4 | 80.2 |
| Naltrexone-morphine pellets | | |
| Naltrexone-morphine cores | (262.4) | (80.2) |
| Ethylcellulose N50 | 22.9 | 7.0 |
| PEG 6000 | 10.6 | 3.2 |
| Eudragit L100-55 | 5.0 | 1.5 |
| DEP | 4.7 | 1.5 |
| Talc | 21.5 | 6.6 |
| Total | 327.1 | 100.0 |

### B. Method of Preparation—

1. Dissolve Ethylcellulose and dibutyl sebacate into ethanol, then disperse talc and magnesium stearate into the solution.
2. Spray the dispersion from 1 onto sugar spheres in a Wurster to form seal-coated sugar spheres (50 μm seal coat).
3. Dissolve Klucel LF and ascorbic acid into 20:80 mixture of water and ethanol. Disperse naltrexone HCl and talc into the solution.
4. Spray the naltrexone dispersion from 3 onto seal-coated sugar spheres from 2 in a Wurster to form naltrexone cores.
5. Dissolve Eudragit RS, sodium lauryl sulfate and dibutyl sebacate into ethanol. Disperse talc into the solution.
6. Spray the dispersion from 5 onto naltrexone cores from 4 in a Wurster to form naltrexone pellets.
7. The Naltrexone pellets are dried at 50° C. for 48 hours.
8. Resulting pellets have a Eudragit RS coat thickness of 150 μm.
9. Dissolve sodium chloride and hypromellose into water.
10. Dissolve hypromellose into 10:90 mixture of water and ethanol. Disperse morphine sulfate into the solution.
11. Spray the solution from 9 followed by the dispersion from 10 onto naltrexone pellets in 7 in a rotor to form naltrexone-morphine cores.
12. Dissolve ethylcellulose, PEG 6000, Eudragit L100-55 and diethyl phthalate into ethanol. Disperse talc into the solution.
13. Spray the dispersion from 12 onto naltrexone-morphine cores in 11 or 12 to form naltrexone-morphine pellets.
14. The pellets are filled into capsules.

### B. In-Vitro Drug Release—

1. Method—USP paddle method at 37° C. and 100 rpm
   1 hour in 0.1 N HCl, then 72 hours in 0.05M pH 7.5 phosphate buffer
Results—Percent of NT released at 73 hours for =0%
2. Method—USP paddle method at 37° C. and 100 rpm
   72 hrs in 0.2% Triton X-100/0.2% sodium acetate/0.002N HCl, pH 5.5
Results—Percent of NT released at 73 hours=0%

### C. In-Vivo Study

This is a single-dose, open-label, two period study in which eight subjects were randomized to receive one dose of PI-1510 under either fasted or fed state during Study Period 1 and alternate fasted or fed state for Study Period 2. Blood samples were drawn prior to dose administration and at 0.5 to 168 hours post-dose. Limits of quantitation are 4.00 pg/mL for naltrexone and 0.250 pg/mL for 6-beta-naltrexol. A summary of the pharmacokinetic measurements is provided in the following tables.

6-beta-Naltrexol levels

PI-1510

| | Fast | Fed |
|---|---|---|
| Tmax (hr) | 45.00 (N = 6) | 57.29 (N = 7) |
| Cmax (pg/mL) | 16.1 | 23.0 |
| AUCₗₐₛₜ (pg*h/mL) | 609.2 | 1057 |
| AUC∞ (pg*h/mL) | 1233 | 1431 (N = 6) |
| T½ (hr) | 17.36 | 17.48 (N = 6) |

Relative Bioavailability to an oral solution (Dose-adjusted)

| | | |
|---|---|---|
| Cmax Ratio (Test/Solution) | 0.44% | 0.68% |
| AUCₗₐₛₜ Ratio (Test/Solution) | 1.97% | 3.42% |
| AUC∞ Ratio (Test/Solution) | 3.86% | 4.49% |

N = 8, unless specified otherwise

It was concluded that PI-1510 and PI-1495 are comparable. The reduction in naltrexone loading in the pellets (from 1.5% in PI-1495 to 0.7% in PI-1510) does not seem to affect NT release. Significant NT sequestering was observed, both at fasting (>96%) and fed states (>95%). The food effect observed was modest in terms of total NT release. However, the lag time was significantly reduced in the presence of food. There were subjects with multiple peaks of release.

### Summary of NT Release from all In-Vivo Studies

BA (Cmax)=Relative bioavailability based on Cmax=Dose-adjusted ratio of Cmax (NT/KNT pellet) to Cmax (NT soln)
BA (AUC last)=Relative bioavailability based on AUC last=Dose-adjusted ratio of AUC last (NT/KNT pellet) to AU
BA (AUC inf)=Relative bioavailability based on AUC inf=Dose-adjusted ratio of AUC inf (NT/KNT pellet)
Total in-vivo cumulative NT release can be extrapolated from BA (AUC inf) calculations from 6-beta-Naltrexol plasma levels

| | BA (Cmax) | BA (AUC last) | BA (AUC inf) |
|---|---|---|---|
| | (%) | (%) | (%) |
| OPTIM. #4 | | | |
| PI-1495 | | | |
| Fast | | | |
| Avg ± SD | 0.5 ± 0.5 | 2.5 ± 2.3 | 2.6 ± 2.4 |
| Range | 0.1-1.4 | 5.9-0.3 | 0.3-5.7 |
| Fed | | | |
| Avg ± SD | 3.0 ± 6.7 | 10.2 ± 19.4 | 11.3 ± 20.0 |
| Range | 0.1-19.4 | 0.2-57.0 | 0.2-55.4 |
| Fed (-Subject 1) | | | |
| Avg ± SD | 0.6 ± 0.9 | 3.6 ± 4.9 | 4.0 ± 5.0 |
| Range | 0.1-2.5 | 0.2-13.8 | 0.2-13.4 |
| PI-1496 | | | |
| Fast | | | |
| Avg ± SD | 1.2 ± 0.9 | 7.1 ± 4.6 | 7.0 ± 4.6 |
| Range | 0.1-2.7 | 0.6-14.2 | 0.6-14.5 |
| Fed | | | |
| Avg ± SD | 2.7 ± 2.9 | 13.4 ± 12.6 | 13.1 ± 12.3 |
| Range | 0.1-7.6 | 0.1-31.6 | 0.4-30.7 |
| OPTIM. #5 | | | |
| PI-1510 | | | |
| Fast | | | |
| Avg | 0.4 | 2.0 | 3.9 |
| Fed | | | |
| Avg | 0.7 | 3.4 | 4.5 |

### Example 3

Kadian NT Formulation #6 (AL-01)

| | | Final |
|---|---|---|
| | 15% | formulation |
| | TPCW | AL-01 |
| Seal-coated Sugar Spheres | | |
| Sugar Spheres (#23-30 mesh) | 11.99 | 11.94 |
| Ethylcellulose NF 50 cps | 2.00 | 1.99 |
| Magnesium Stearate NF | 0.80 | 0.80 |
| Dibutyl Sebacate NF | 0.20 | 0.20 |
| Talc USP (Suzorite 1656) | 5.00 | 4.98 |
| Naltrexone HCl Core | | |
| Seal-coated Sugar Spheres | | (19.90) |
| Naltrexone Hydrochloride USP | 0.73 | 0.72 |
| Hydroxypropyl Cellulose NF | 0.14 | 0.14 |
| Ascorbic Acid USP | 0.07 | 0.07 |
| Talc USP (Suzorite 1656) | 0.34 | 0.34 |
| Naltrexone HCl Intermediate Pellet | | |
| Naltrexone HCl Core | | (21.17) |
| Ammonio Methacrylate Copolymer Type B NF | 6.26 | 6.23 |
| Sodium Lauryl Sulfate NF | 0.22 | 0.22 |
| Dibutyl Sebacate NF | 0.63 | 0.62 |
| Talc USP (Suzorite 1656) | 6.08 | 6.05 |
| Naltrezone HCl Finished Pellet | | |
| Naltrexone HCl Intermediate Pellet | | (34.29) |
| Ammonio Methacrylate Copolymer Type B NF | 9.89 | 9.85 |
| Sodium Lauryl Sulfate NF | 0.34 | 0.34 |
| Dibutyl Sebacate NF | 0.99 | 0.98 |
| Talc USP (Suzorite 1656) | 9.71 | 9.67 |
| NaCl Overcoated Naltrexone HCl Pellet | | |
| Naltrexone HCl Finished Pellet | | (55.13) |
| Sodium Chloride USP | 3.75 | 3.73 |
| Hydroxypropyl Cellulose NF | 0.42 | 0.41 |
| MS Cores with Sequestered Naltrexone HCl | | |
| NaCl Overcoated Naltrexone HCl Pellet | | (59.28) |
| Morphine Sulfate USP | 18.11 | 18.03 |
| Hydroxypropyl Cellulose NF | 1.42 | 1.42 |
| MS Extended-release with Sequestered | | |
| Naltrexone HCl Pellet | | |
| MS Cores with Sequestered Naltrexone HCl | | (78.73) |
| Component (a): ethylcellulose NF (50 cps) | 7.40 | 7.36 |
| Component (c): polyethylene glycol NF (6000) | 3.42 | 3.40 |
| Component (b): methacrylic acid copolymer NF | 1.60 | 1.60 |
| (Type C, Powder) | | |
| Diethyl Phthalate NF (plasticizer) | 1.53 | 1.53 |
| Talc USP (Suzorite 1656) (filler) | 6.98 | 7.38 |
| Total | 100.0 | 100.0 |

In certain embodiments, components (a), (b) and/or (c) may be included as described below:
(a) preferably a matrix polymer insoluble at pH of about 1 to about 7.5; preferably ethylcellulose; preferably at least 35% by weight of a+b+c;
(b) preferably an enteric polymer insoluble at pH of about 1 to about 4 but soluble at pH of about 6 to about 7.5; preferably methacrylic acid-ethyl acrylate copolymer (methacrylic acid copolymer type C) preferably about 1 to about 30% of a+b+c; and,
(c) compound soluble at a pH from about 1 to about 4; preferably polyethylene glycol with a molecular weight from about 1700 to about 20,000; preferably from about 1% to about 60% by weight of a+b+c.

### C. Method of Preparation

1. Ethylcellulose and Dibutyl Sebacate were dissolved into Alcohol SDA3A. Talc and Magnesium Stearate were then dispersed into the solution. The percent solid of the dispersion was 20%.
2. The dispersion from 1 was sprayed onto Sugar Spheres in a Wurster to form Seal-coated Sugar Spheres (approx. 50 μm seal coat).
3. Hydroxypropyl Cellulose and Ascorbic Acid were dissolved into a 20:80 mixture of Water and Alcohol SDA3A. Naltrexone HCl and Talc were then dispersed into the solution. The percent solid of the dispersion is 20.4%.
4. The Naltrexone HCl dispersion from 3 was sprayed onto Seal-coated Sugar Spheres from 2 in a Wurster to form Naltrexone HCl cores.
5. Ammonio Methacrylate Copolymer, Sodium Lauryl Sulfate and Dibutyl Sebacate were dissolved into a 22:78 mixture of Water and Alcohol SDA3A. Talc was dispersed into the solution. The percent solid of the dispersion was 20%.
6. The dispersion from 5 was sprayed onto Naltrexone HCl cores from 4 in a Wurster to form Naltrexone HCl Intermediate Pellets.
7. The Naltrexone HCl Intermediate Pellets were dried in an oven at 50° C. for 24 hours.
8. Ammonio Methacrylate Copolymer, Sodium Lauryl Sulfate and Dibutyl Sebacate were dissolved into a 22:78 mixture of Water and Alcohol SDA3A. Talc was dispersed into the solution. The percent solid of the dispersion was 20%.
9. The dispersion from 8 was sprayed onto Naltrexone HCl Intermediate Pellets from 7 in a Wurster to form Naltrexone HCl Finished Pellets.
10. The Naltrexone HCl Finished Pellets were dried in an oven at 50° C. for 24 hours.
11. The resulting pellets had a pellet coat thickness of approximately 150 μm.
12. Sodium Chloride (NaCl) and Hydroxypropyl Cellulose were dissolved into Water. The percent solid in the solution was 6%.
13. The Sodium Chloride solution from 12 was sprayed onto Naltrexone HCl Finished Pellets from 10 in a Wurster to form Sodium Chloride (NaCl) Overcoated Naltrexone HCl Pellets.
14. Hydroxypropyl Cellulose was dissolved into Alcohol SDA3A, and Morphine Sulfate dispersed into the solution. The percent solid in the dispersion was 24.4%.
15. The Morphine Sulfate dispersion from 14 was sprayed onto NaCl Overcoated Naltrexone HCl Pellets in 13 in a rotor to form Morphine Sulfate Cores with Sequestered Naltrexone HCl.
16. Ethylcellulose, Polyethylene Glycol, Methacrylic Acid Copolymer and Diethyl Phthalate were dissolved into Alcohol SDA3A. Talc was dispersed into the solution. The percent solid in the dispersion was 14.3%.
17. The Dispersion from 16 was sprayed onto Morphine Sulfate Cores with Sequestered Naltrexone NCI in 15 to form Morphine Sulfate Extended-release with Sequestered Naltrexone HCl Pellets.
18. The pellets were filled into capsules.

### Example 4

Proprietary formulations being developed by Alpharma Pharmaceuticals LLC, such as those described herein, contain morphine and naltrexone. The formulation technology allows the morphine component of the drug product to be released in a controlled fashion, while sequestering the naltrexone component so it is not released in clinically significant quantities during normal dosing conditions. If any attempt is made to defeat or manipulate the formulation developed by Alpharma—such as crushing—the normally sequestered naltrexone will be released thereby actively antagonizing the effects of the morphine upon administration or dosing.

In an ongoing proof of abuse deterrent concept study, 30 nondependent, recreational opioid drug users received single oral dose administrations of ALO-01 whole/intact, ALO-01 crushed, morphine sulfate IR oral solution, and placebo in a 4-way crossover triple dummy trial. The primary objective of the study is to determine the relative effect of naltrexone antagonism on drug-liking and euphoria when the product was abused by crushing and consumed orally. The rationale for the current study is to simulate and characterize the effect of naltrexone on the pharmacodynamic (PD) profile of morphine if the oral dosage form was crushed and injected. The desired effect of the naltrexone dose is a reduction of the subjective drug effects associated with administration of morphine alone. Experience in other abuse liability trials has lead to the selection of the Drug Effects Questionnaire (DEQ) Question #5, “How high are you now?”, as the most sensitive indicator of euphoric response and will therefore be the primary efficacy as well as pharmacodynamic endpoint of this trial. The Cole/ARCI Stimulation Euphoria scale will also be used to assess euphoric response.

### Primary Objective

To determine the relative drug-liking and euphoric effects of IV morphine alone to IV morphine combined with IV naltrexone, as reflected in pharmacodynamic measures following single IV bolus doses.

### Secondary Objectives

To determine the relative drug-liking and euphoric effects of IV morphine alone and IV morphine combined with IV naltrexone to placebo as reflected in pharmacodynamic measures following single IV bolus doses.
To determine the relative effect of IV morphine alone compared to IV morphine plus IV naltrexone on end-tidal CO₂ (EtCO₂) as measured by capnography.
To determine the relative effect of morphine alone compared to IV morphine plus IV naltrexone on pupillometry.
To evaluate the safety of single doses of IV morphine alone and IV morphine combined with IV naltrexone
To assess the pharmacokinetics of plasma morphine, naltrexone, and 6β-naltrexol following intravenous administrations of morphine alone and morphine with naltrexone.
To explore plasma naltrexone concentrations associated with 25%, 50%, 75%, and 100% (ie, no different from placebo) decreases in drug-liking and euphoria over time from maximum effects of IV morphine alone. Plasma naltrexone concentrations associated with changes in other pharmacodynamic measurements (EtCO2 and pupillometry) relative to IV morphine alone may also be explored.

Protocol ALO-01-07-106 is a single-center, randomized, double-blind cross-over trial in non-dependent opioid-preferring male subjects to characterize the effect of naltrexone on the euphorogenic effects of morphine as reflected in the subjective responses to the DEQ and Cole/ARCI.

Prior to entering the trial each subject must complete all screening procedures and report to the clinic for a Naloxone Challenge Test to rule out subjects who are physically dependent upon opioids. Each subject that successfully completes the Naloxone Challenge Test will undergo a Drug Discrimination Phase.

During the three-day in-patient Drug Discrimination Phase, subjects will be randomized to receive either placebo or 10 mg of morphine on the first and third days of this phase. Subjects will be asked to answer a battery of questions using the DEQ and Cole/ARCI at designated time points following each dose.

At the conclusion of the Drug Discrimination Phase, the blind will be broken for each subject and the investigator will determine if the subject is able to successfully discriminate between morphine and placebo. Subjects who are able to discriminate between morphine and placebo will stay at the research site for a one-day washout and then begin the Treatment Phase of the study.

During the Treatment Phase, subjects will participate in three treatment procedures listed below. Randomization will occur following successful completion of the Drug Discrimination Phase. All test drug products will be intravenously administered and will be supplied by the Lifetree pharmacist after blinding. All subjects will be randomized to three sequential treatment doses using a crossover design. Subjects will receive one dose on each dosing day of this phase in a double-blinded; cross-over manner (with a 6-day outpatient washout in between). Subjects will be randomized to receive each of the following dosing schedules in various sequences:
a single 30 mg IV dose of morphine+a single IV dose of naltrexone placebo,
a single 30 mg IV dose of morphine+a single 1.2 mg IV dose of naltrexone,
a single IV dose of morphine placebo+a single IV dose of naltrexone placebo.

Subjects will be asked to answer a battery of questions using the DEQ and Cole/ARCI at designated time points following each dose. Blood samples will be drawn for morphine, naltrexone, and 6β-naltrexol pharmacokinetic measurements.

Study Procedures.

Prior to any study-related activities, the Informed Consent Form must be signed and dated by the subject. The format and content of the Informed Consent Form will be agreed upon by the Principal Investigators(s) and the appropriate Institutional Review Board (IRB). The signed and dated Informed Consent Form must be retained by the Investigator in the subject's file.

Screening Visit.

The following will be completed within 28 days prior to admission to the study center:
Informed Consent (written consent will be obtained prior to conducting any screening activities)
Review of Inclusion and Exclusion Criteria
Medical history
Record concomitant medications
Pulse oximetry and vital sign measurements are taken (after a 3-minute sitting period).
Brief physical examination, including measurement of height and weight
A 12-lead ECG
Laboratory assessments, including serum chemistry, hematology, urinalysis, Hepatitis B, C, and HIV antibodies
Urine drug screen (Subjects must test negative for Benzodiazepines. Amphetamines, Cocaine and opioids [includes methadone]). If the subject tests positive for any of these, they may return to the study center prior to expiration of the screening window to have a repeat urine drug screen.

Naloxone Challenge Test (Day 0).

Subjects will check into the study center on the morning of Day 0 and will remain confined to the study center through completion of the first dose in the Treatment Phase unless discharged for cause. On Day 0 the following procedures will be performed:
Confirmation of inclusion/exclusion criteria
Urine drug screen (Subjects must test negative for Benzodiazepines. Amphetamines, Cocaine and opioids [includes methadone])
Review of concomitant medications
Pulse oximetry and vital signs
Ethanol breath test
Following Day 0 procedures, subjects will undergo an intravenous Naloxone Challenge to rule out physically dependent individuals. The procedure for the Naloxone Challenge is as follows:
Subjects will receive a total of 0.8 mg intravenous naloxone.
A dose of 0.2 mg will be injected initially while the subject is observed for signs or symptoms of withdrawal.
If there is no evidence of withdrawal occurring in 30 seconds, the remaining 0.6 mg of naloxone will be injected and the subject will be observed for 20 minutes for signs and symptoms of withdrawal.
Subjects demonstrating evidence of withdrawal will not be eligible for further participation in the trial and the Discharge Procedures will be completed. The subject will be released from the study center when medically stable as determined by medical personnel at the study site.
Subjects NOT evidencing withdrawal will remain in the study center for the remainder of the day and overnight for continuing participation in the trial.
Subjects will report adverse events

Drug Discrimination Phase (Day 1 and Day 3).

Subjects passing the Naloxone Challenge will enter the Drug Discrimination Phase. On Day 1 and Day 3:
Subjects will be randomized to receive 10 mg morphine or placebo IV. Vital signs and pulse oximetry will be taken PRIOR to dosing and at 1, 2, 4, 8, and 12 hours following dosing.
Record concomitant medications
Subjects will be administered test product IV. Subjects will receive one double-blind injection on Day 1 and one double-blind injection on Day 3.
Subjects will complete the DEQ scales immediately before dosing (t=0) and at 5, 30, 60, 90, 120, 180, 240, and 300 minutes after dosing.
Subjects will complete the Cole/ARCI Stimulation Euphoria Scale immediately before dosing (t=0) and at 5, 30, 60, 90, 120, 180, 240, and 300 minutes after dosing.
Subjects will report all adverse events.
NOTE: The methods for preparation and dosing of morphine in this study will result in rapid release and uptake of morphine such that subjects may experience some symptoms of opioid toxicity. Therefore, naloxone will be readily available at all times for IV administration. Following completion of ALL study-related procedures on Day 3, subjects will remain in the study center until the DEQ data is reviewed by an investigator and a determination made regarding the subject's suitability to continue into the Treatment Phase. The study blind will be broken for the Drug Discrimination Phase only at this time to assist the investigator in determining the subject's eligibility to continue in the study. Those subjects who, in the investigator's opinion, were unable to distinguish between morphine test product and placebo will be classified as an early terminated subject and discharged from the research center after completing discharge procedures. Those subjects who, in the investigator's opinion, were able to distinguish between morphine test product and placebo will be allowed to continue participation in the Treatment Phase of the study and will stay at the research site for a washout day (Day 4).

Treatment Phase (Day 5 through Day 19).

Subjects successfully completing all study procedures on Day 1 and Day 3 of the Drug Discrimination Phase and, in the investigator's opinion, were able to distinguish between morphine and placebo will be eligible for entry into the Treatment Phase. NOTE: The methods for preparation and dosing of morphine in this study will result in rapid release and uptake of morphine. Subjects may experience some symptoms of opioid toxicity; therefore, naloxone will be readily available at all times for IV administration.

Procedures During Treatment Phase:
Subjects will dose with test product on Days 5, 12, and 19, with an outpatient washout on
Days 6-11 and Days 13-18. Subjects will be randomized to receive 30 mg morphine alone, or 30 mg morphine with 1.2 mg naltrexone, or placebo.
Vital signs and pulse oximetry will be taken PRIOR to dosing and at 1, 2, 4, 8, and 12 hours following dosing.
Record concomitant medications
Subjects will be administered test product IV.
Subjects will complete the DEQ scales immediately before dosing at baseline (t=0) and at 5, 15, 30, 45, 60, 90, 120, 150, 180, 210, 240, 270, 300, 360, 480, 720, and 1440 min after dosing.
Subjects will complete the Cole/ARCI Stimulation Euphoria Scale immediately before dosing at baseline (t=0) and at 5, 15, 30, 45, 60, 90, 120, 150, 180, 210, 240, 270, 300, 360, 480, 720, and 1440 min after dosing.
Pupillometry (one eye) at baseline (t=0) and at 5, 15, 30, 45, 60, 90, 120, 150, 180, 210, 240, 270, 300, 360, 480, 720, and 1440 min after dosing.
Blood sample being drawn for plasma morphine, naltrexone, and 6β-naltexol determinations at baseline (t=0) and at 5, 15, 30, 45, 60, 90, 120, 150, 180, 210, 240, 270, 300, 360, 480, 720, and 1440 min after dosing.
All adverse events will be recorded.
The nominal times expressed in minutes correspond to: 0.083, 0.25, 0.5, 0.75, 1, 1.5, 2, 2.5, 3, 3.5, 4, 4.5, 5, 6, 8, 12, and 24 hours after dosing.
On “re-check-in” days (Days 12 and 19), subjects will undergo the following assessments prior to dosing with the next study drug in their randomized sequence:
Urine drug screen (Subjects must test negative for Benzodiazepines. Amphetamines, Cocaine and opioids [includes methadone])
Record concomitant medications
Ethanol breath test
Record adverse events.

Discharge Procedures.

Following completion of ALL study-related procedures, or in the event of an early termination, subjects will be eligible for discharge from the study site when the following are completed:
At least 24 hours have passed since their last dose of test product;
Record concomitant medications and adverse events;
Pulse oximetry and vital sign measurements are taken (after a 3-minute sitting period);
Brief physical examination to confirm subject is medically stable;
A 12-lead ECG;
Laboratory assessments, including serum chemistry, hematology, and urinalysis.
Upon discharge, subjects will be instructed to avoid any opioids for at least 72 hours following administration of the test product.

Clinical Laboratory Tests.

All clinical laboratory tests will be performed by the study center at a local laboratory. The following clinical lab tests will be performed at screening:
Hematology: white blood cell count with differential, red blood cell count, hemoglobin, hematocrit, and platelet count;
Serum Chemistry: glucose, sodium, potassium, chloride, bicarbonate, blood urea nitrogen (BUN), creatinine, uric acid, phosphorus, calcium, total protein, albumin, globulin, alkaline phosphatase, alanine transaminase (ALT), aspartate transaminase (AST), total bilirubin, and lactose dehydrogenase (LDH);
Urinalysis: color, specific gravity, pH, protein, sugar, ketones, and occult blood;
Hepatitis B & C antigen and HIV antibody (at Screening only)
Urine drug screen (iCUP) will be performed on site by the study center: amphetamines, barbiturates, benzodiazepines, cocaine, opiates, and cannabinoids;

The following clinical lab tests will be performed on Day 0 before the Naloxone Challenge:
Urine drug screen (iCUP): amphetamines, barbiturates, benzodiazepines, cocaine, opiates, and cannabinoids.

The following clinical lab tests will be performed at discharge from the study or early termination and will be sent to a local laboratory:
Hematology: white blood cell count with differential, red blood cell count, hemoglobin, hematocrit, and platelet count;
Serum Chemistry: glucose, sodium, potassium, chloride, bicarbonate, blood urea nitrogen (BUN), creatinine, uric acid, phosphorus, calcium, total protein, albumin, globulin, alkaline phosphatase, alanine transaminase (ALT), aspartate transaminase (AST), total bilirubin, and lactose dehydrogenase (LDH);
Urinalysis: color, specific gravity, pH, protein, sugar, ketones, and occult blood.

### Preparation of Plasma Samples for Pharmacokinetics

Blood Sample Collection.

Approximately 477 ml of blood will be drawn during this study. Approximately 45 ml will be drawn for screening and end of study labs and 432 ml will be drawn for 54 PK draws (2 tubes each).

For Morphine Analysis in Plasma:

Blood samples for morphine analysis will be collected in appropriately labeled, evacuated blood collection tubes (3 mL), containing sodium heparin as the anticoagulant.

For Naltrexone and 6β-naltrexol Analysis in Plasma:

Blood samples for naltrexone analyses will be collected in appropriately labeled, evacuated blood collection tubes (5 mL), containing K₂-EDTA as the anticoagulant.

Blood Sample Handling.

Immediately after collection, the filled blood collection tubes will be gently inverted several times to insure that the anticoagulant is thoroughly mixed with the blood. Blood samples (approximately 8 mL), collected for both morphine and naltrexone/6β-naltrexol assays, are then to be pooled and split into 2 aliquots and then cooled in an ice bath. Within 45 minutes after collection, blood samples will be centrifuged at 4° C. for 10 minutes at 3,000 RPM. Plasma will be harvested within 30 minutes from the centrifuged samples using pipettes and transferred, in equally sized split samples, into appropriately labeled polypropylene screw top transfer tubes. The harvested plasma samples will be immediately transferred to a freezer, where they will be frozen in the upright position and maintained at −20±10° C. or colder until they are assayed. Split samples will be kept separate, so that there are two complete sets of samples (one primary and one back-up sample). The samples are to be stored in suitably labeled tubes pending assay.

A summary of the study is shown below:
Inpatient (drug discrimination phase): admit on day 0 with naloxone challenge, dose on day 1, washout on day 2, dose on day 3, washout on day 4, dose on day;
Outpatient (treatment phase): washout days 6-11, dose day 12, washout days 13-18, dose day 19.

Schedule of Events

| | Screening¹ | Naloxone | Drug Discrimination Phase | | | | Treatment Phase | | | | | Discharge |
|---|---|---|---|---|---|---|---|---|---|---|---|---|
| | −28 days | Challenge | (Inpatient, Double-blind, Cross-over) | | | | (Out-patient, Double-blind, Cross-over) | | | | | Day 20 or |
| | to | (Inpatient) | Day 1 | Day 2 | Day 3 | Day 4 | Day 5 | Days 6-11 | Day 12 | Days 13-18 | Day 19 | Early |
| Procedures | −1 day | Day 0 | (Dose) | (Washout) | (Dose) | (Washout) | (Dose) | (Washout) | (Dose) | (Washout) | (Dose) | Term |
| Written | X | | | | | | | | | | | |
| Informed | | | | | | | | | | | | |
| Consent | | | | | | | | | | | | |
| Inclusion/ | X | X | | | | | | | | | | |
| Exclusion | | | | | | | | | | | | |
| Pulse Oximetry | X | X | X⁷ | | X⁷ | | X⁷ | | X⁷ | | X⁷ | X |
| and Vital | | | | | | | | | | | | |
| Signs² | | | | | | | | | | | | |
| Brief Physical | X³ | | | | | | | | | | | X |
| Examination | | | | | | | | | | | | |
| Medical | X | | | | | | | | | | | |
| History | | | | | | | | | | | | |
| 12 lead ECG | X | | | | | | | | | | | X |
| Clinical | X | | | | | | | | | | | X |
| Laboratory | | | | | | | | | | | | |
| Tests⁴ | | | | | | | | | | | | |
| Concomitant | X | X | X | X | X | | X | | X | | X | X |
| Medications | | | | | | | | | | | | |
| Urine Drug | X | X | | | | | | | X | | X | |
| Screen | | | | | | | | | | | | |
| Ethanol Breath | | X | | | | | | | X | | X | |
| Test | | | | | | | | | | | | |
| Naloxone | | X | | | | | | | | | | |
| Challenge⁵ | | | | | | | | | | | | |
| Drug Dosing | | | X | | X | | | | | | | |
| (10 mg | | | | | | | | | | | | |
| Morphine | | | | | | | | | | | | |
| or Placebo)⁶ | | | | | | | | | | | | |
| Drug Dosing | | | | | | | X | | X | | X | |
| (30 mg | | | | | | | | | | | | |
| morphine, | | | | | | | | | | | | |
| 1.2 mg | | | | | | | | | | | | |
| naltrexone, | | | | | | | | | | | | |
| placebo)⁶ | | | | | | | | | | | | |
| Administer | | | X¹⁰ | | X¹⁰ | | X⁸ | | X⁸ | | X⁸ | |
| DEQ & | | | | | | | | | | | | |
| Cole/ARCI | | | | | | | | | | | | |
| Euphoria | | | | | | | | | | | | |
| Scales | | | | | | | | | | | | |
| End-tidal CO₂ | | | | | | | X⁸ | | X⁸ | | X⁸ | |
| Pupillometry | | | | | | | X⁸ | | X⁸ | | X⁸ | |
| PK Sampling | | | | | | | X⁹ | | X⁹ | | X⁹ | |
| Adverse Events | | X | X | X | X | X | X | | X | | X | X |

¹The Screening Period is defined as the period between the Screening Visit and the Day 0 Visit.
²Vital signs include blood pressure, heart rate, and respiratory rate (Temperature taken only at screening visit)
³Record height and weight at Screening Visit only
⁴Clinical laboratory tests include serum chemistry, hematology, and urinalysis; hepalitis B and C antigens and HIV antibody at Screening only.
⁵Subjects may receive up to a total of 0.8 mg intravenous naloxone. 0.2 mg will be injected initially while the subject is observed for signs or symptoms of withdrawal. If there is no evidence of withdrawal occurring in 30 seconds the remaining 0.6 mg of naloxone will be injected and subject observed for 20 minutes for signs and symptoms of withdrawal. Subjects demonstrating evidence of withdrawal will not be eligible for further participation in the trial and the end of study procedures will be completed. The subject will be released from the study center when medically stable as determined by medical personnel at the study site Subjects NOT evidencing withdrawal symptoms will be housed in the study center for continuing participation in the trial
⁶Test product will be administered IV
⁷Collect Vital Signs and pulse oximetry measurements prior in dosing and at 1, 2, 4, 8, and 12 hours post dose
⁸Administered at baseline immediately before dosing (t = 0) and immediately prior to each PK sample at 5, 15, 30, 45, 60, 90, 120, 150, 180, 210, 240, 270, 300, 360, 480, 720, and 1440 min after dosing
⁹Collect blood samples at baseline immediately before dosing (t = 0) and at 5, 15, 30, 45, 60, 90, 120, 150, 180, 210, 240, 270, 300, 360, 480, 720, and 1440 min after dosing.
¹⁰Administer at baseline immediately before dosing (t = 0) and at 5, 30, 60, 90, 120, 180, 240, and 300 minutes after dosing

The population for this trial consists of non-dependent, opioid-preferring male recreational drug abusers. Each subject enrolled in this trial must meet the following criteria:
The subject is a male between 18 and 50 years old, inclusive.
The subject has a body mass index (BMI) within 18-33 kg/m².
The subject is in general good health as determined by the medical history, physical exam, laboratory tests, and electrocardiogram (ECG).
The subject is a recreational drug user who is NOT physically dependent on opioids but has used prescription opioids to achieve a “high” on at least 5 occasions in the last 12 months. Subjects who use multiple drugs should express a preference for opioids.
The subject is able to speak, read, and understand English sufficiently to understand the nature of the study, to provide written informed consent, and complete all study assessments.
The subject is willing and able to comply with all testing requirements defined in the protocol.
   Subjects meeting any of the following criteria will be excluded:
The subject has any relevant deviations from normal in physical examination, ECG, or clinical laboratory tests, as evaluated by the investigator.
The subject has had a clinically significant illness within 30 days preceding entry into this study.
The subject has a history of significant neurological, hepatic, renal, endocrine, cardiovascular, gastrointestinal, pulmonary, or metabolic disease.
The subject has a known allergy or history of hypersensitivity to morphine, other opioids, or similar compounds.
The subject has used any prescription medication within 14 days or any over-the-counter (OTC) medication, alcohol, or grapefruit and grapefruit juice within 48 hours of dosing or intends to use any prescription or OTC medication during the study that may interfere with the evaluation of study medication.
The subject has participated in another drug study within 30 days prior to initiation of this study.
Subjects who have made a donation of blood or a significant blood loss within 60 days prior to the first dose of study drug.
Subjects who have made a plasma donation within 7 days prior to the study.
Subjects with screening hemoglobin less than 12.0 g/dL.
The subject is currently in treatment for substance abuse or who has completed a substance abuse treatment program within 90 days.
The subject has completed a substance abuse program and has NOT relapsed.
The subject has a positive urine drug screen (UDS) for amphetamines, barbiturates, benzodiazepines, cocaine, or opiates upon presentation for admission to the clinic. Subjects may return for re-drug screen to the clinic for re-evaluation and inclusion in the study.
The subject is unable or unwilling, in the opinion of the investigator, to comply with all study procedures and cooperate fully with Lifetree Clinical Research staff.

In accordance with the protocol, subjects will be terminated at the end of the Naloxone Challenge Phase if they exhibit signs of opioid withdrawal and at the end of the Discrimination Phase if in the judgment of the PI they are unable to discriminate morphine from placebo. Subjects may also choose to discontinue test product or study participation at any time, for any reason, and without prejudice. Upon study termination, Discharge Procedures must be followed before subject is discharged and the reason for early termination, if applicable, must be documented in the source documents and Case Report Forms (CRFs).

Subjects will undergo an intravenous Naloxone Challenge in which they will receive a total of 0.8 mg intravenous naloxone. A dose of 0.2 mg will be injected IV initially while the subject is observed for signs or symptoms of withdrawal. If there is no evidence of withdrawal occurring in 30 seconds the remaining 0.6 mg of naloxone will be injected and the subject will be observed for 20 minutes for signs and symptoms of withdrawal. For the Drug Discrimination Phase, subjects will be randomized to receive 10 mg morphine or placebo IV. Subjects will receive one double-blind injection on Day 1 and one double-blind injection on Day 3. During the Treatment Phase, subjects will receive one dose on each dosing day in a double-blinded, crossover manner (with a 6 day outpatient washout in between). Subjects will be randomized to receive each of the following dosing schedules in various sequences: (1) a single 30 mg IV dose of morphine+a single IV naltrexone placebo, (2) a single 30 mg IV dose of morphine+a single 1.2 mg dose of IV naltrexone, and (3) a single IV dose of morphine placebo+a single IV naltrexone placebo.

Use of prescription medications will be prohibited for 2 weeks before admission to the study center (Day 0) and during the study. Use of OTC medication will be prohibited for 48 hours before admission to the study center (Day 0) and during the study. Use of alcohol, grapefruit, and grapefruit juice is prohibited 48 hours before admission to the study center (Day 0) and through the duration of the study.

Subjects are monitored for compliance with study inclusion/exclusion criteria through a urine drug screen at screening day, before the Naloxone Challenge (Day 0), and upon re-check in days 12 and 19. They also take an ethanol breath test on Day 0 and on re-check-in-days 12 and 19. The Naloxone Challenge is a measure of whether they are physically dependent on opioids.

Drug products are administered as IV injections. Subjects will undergo an intravenous Naloxone Challenge in which they will receive a total of 0.8 mg intravenous naloxone. A dose of 0.2 mg will be injected IV initially while the subject is observed for signs or symptoms of withdrawal. If there is no evidence of withdrawal occurring in 30 seconds the remaining 0.6 mg of naloxone will be injected.

For the Drug Discrimination Phase, subjects will be randomized to receive 10 mg morphine or placebo IV. Subjects will receive one double-blind injection on Day 1 and one double-blind injection on Day 3. During the Treatment Phase, subjects will receive one dose on each dosing day:
a single 30 mg IV dose of morphine+a single IV dose of naltrexone placebo,
a single 30 mg IV dose of morphine+a single 1.2 mg IV dose of naltrexone,
a single IV dose of morphine placebo+a single IV dose of naltrexone placebo.
   All subjects will be dosed according to the study procedures outlined in the INVESTIGATIONAL PLAN section of this protocol.

The DEQ and the Cole/ARCI Euphoria subscale will be used to assess efficacy as well as pharmacodynamics. The following is a description of each efficacy measurement:
Drug Effects Questionnaire: This questionnaire contains 9 items, each presented as a 100 mm VAS.
Cole/ARCI Euphoria Subscale: This scale consists of 15 statements that subjects score using a 4-point scale (0-3), where 0=false, 1=more false than true, 2=more true than false, and 3=true. The total score is calculated by adding the individual scores and the total possible score is 45.
   Each measurement will take place immediately prior to each PK sample on dosing days in the double-blind Treatment Phase.

This study will evaluate the euphoria-blocking effects of naltrexone hydrochloride when combined with morphine sulfate. The pharmacodynamic effect will be evaluated by using the Drug Effects Questionnaire (DEQ) and the Cole/ARCI Stimulation Euphoria Scale. The primary criterion for evaluating the euphoria blocking effects of naltrexone will be question 5, “how high are you?” on the DEQ.

Approximately 76 subjects will sign consent and screen for the study. Approximately 40 subjects will participate in the naloxone challenge. Approximately 34 subjects will be enrolled into the drug discrimination phase of the study with 24 subjects completing the study in its entirety. The sample size was not determined on the basis of statistical calculation but as a suitable sample size based on previous studies of similar design to detect differences between the two dosing groups. Analysis groups are defined as follows:
The double-blind safety population includes all subjects that received at least one dose of study drug during the double-blind Treatment Phase.
The PK population will be subjects who completed at least one study treatment period in the double-blind Treatment Phase.
The Evaluable PK population will be subjects who completed at least two study treatment periods in the double-blind Treatment Phase.
The PD population will include subjects who received at least one study treatment in the double-blind Treatment Phase and provided at least one subsequent efficacy or PD assessment during the double-blind Treatment Phase.
The Evaluable PD population will include subjects who completed at least two study treatment periods in the double-blind Treatment Phase.

The results of the DEQ question #5, “low high are you?” will constitute the primary pharmacodynamic (PD) endpoint. Other PD assessments include the other subscales of the DEQ, the Cole/ARCI Euphoria subscale, EtCO₂ levels determined by non-invasive capnography, and pupillometry. The maximum scores for each efficacy and PD assessment within a period will be used for analysis. Each maximum efficacy and PD score will be analyzed using a linear mixed model with fixed effects for sequence, period, and treatment arm, and a random effect for subject nested in sequence, will be used. Least squares means along with 90% confidence intervals will be provided for each treatment arm and for all pair-wise contrasts between treatment arms. In addition to analyzing the maximum scores, the AUE will be calculated for each treatment period and treatment arm.

The PK analyses will be based on all available post-dosing PK data. For each subject, the pharmacokinetic parameters will be determined by using a non-compartmental approach. Summary statistics for plasma concentrations of morphine, naltrexone, and 6β-naltrexol will be calculated by time and dose. In addition, PK and PD parameters will be summarized using descriptive statistics (n, arithmetic mean, median, standard deviation (SD), minimum, maximum, coefficient of variation, geometric mean [Eₘₐₓ, AUC, AUE and C₀ only]). For the purpose of plotting the data, plasma concentration values that are below the limit of quantification (BLQ) imbedded between two measurable concentrations will be set to missing, however, BLQ's occurring after the last measurable plasma concentration will be set to zero. For the purpose of the noncompartmental pharmacokinetic analysis, all BLQ's occurring after the first measurable plasma concentration will be set to missing. The following pharmacokinetic parameters will be calculated:
The anticipated initial plasma drug concentration (C₀) given as the intercept on the plasma concentration axis when the line is extrapolated back to time 0.
Area under the plasma concentration-time curve (AUC) from time zero to 2, 8, and 24 hours post dose (AUC₀₋₂, AUC₀₋₈, AUC₀₋₂₄), computed using the linear trapezoidal rule.
The area under the plasma concentration versus time curve from time 0 to infinity. (AUC_{inf}) is calculated as the sum of AUC₀₋ₜ plus the ratio of the last measurable plasma concentration to the elimination rate constant.
Apparent first-order terminal rate constant (kₑₗ) calculated from a semi-log plot of the plasma concentration versus time curve. The parameter will be calculated by linear least-squares regression analysis using the maximum number of points in the terminal log-linear phase (e.g. three or more non-zero plasma concentrations).
Apparent first-order terminal half-life (t_{1/2}) will be calculated as 0.693/kₑₗ.
Steady state volume of distribution (Vₛₛ) computed using the linear trapezoidal rule as dose*AUMC)/AUC².
Total plasma clearance (CLₜ) computed as dose/AUC.

The following pharmacodynamic parameters will be calculated:
The maximum effect (Eₘₐₓ) determined by direct observation of the data
The time of maximum effect (TEₘₐₓ) determined by direct observation of the data
The area under the effect curve (AUE) from time zero to 2, 8, and 24 hours post dose (AUE₀₋₂, AUE₀₋₈, and AUE₀₋₂₄), computed using the linear trapezoidal rule.

### Results

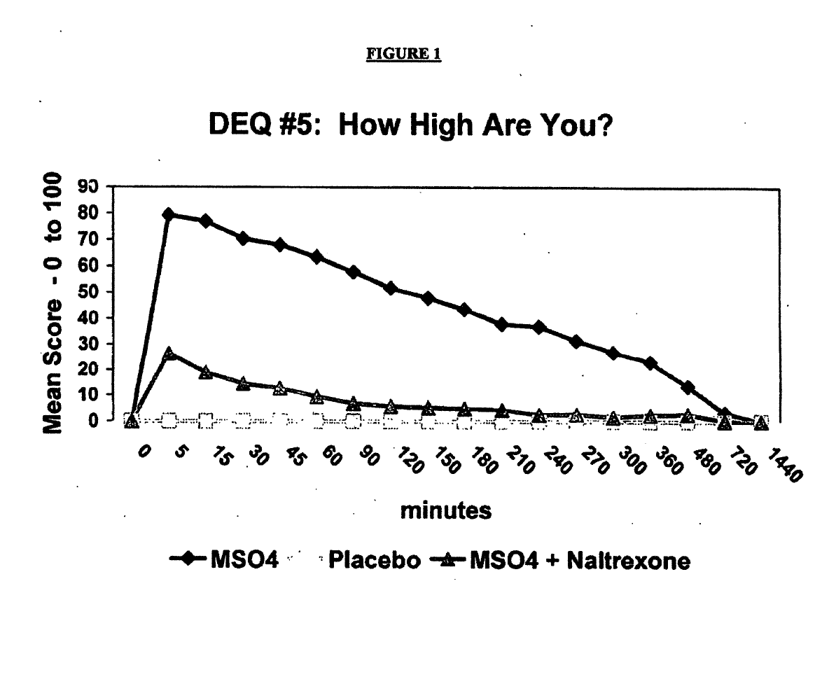

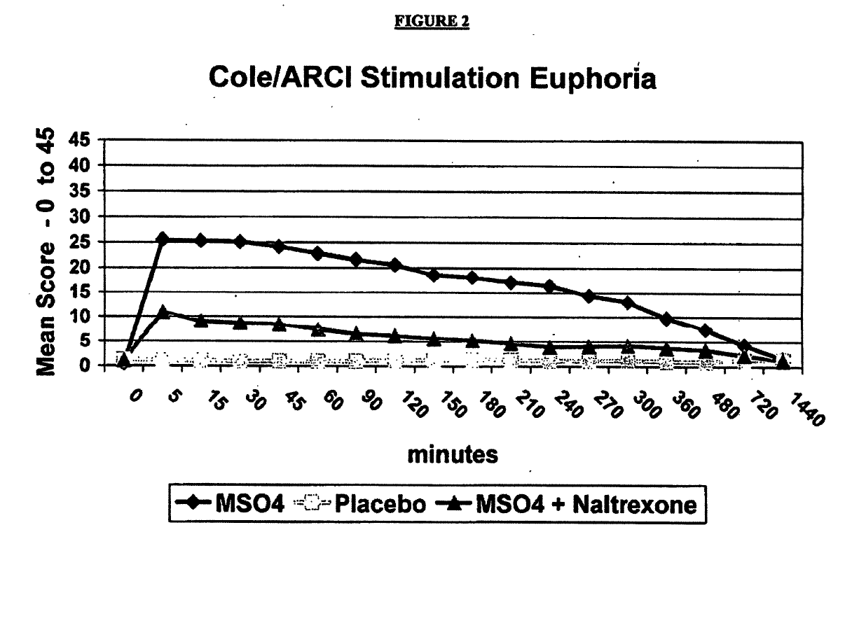

A summary of the results of the primary endpoint are shown in FIG. 1. A summary of the results of the secondary endpoint is shown in FIG. 2. As shown therein, the opioid antagonist administered in these studies inhibited the activity of the opioid agonist.

While the present invention has been described in terms of the preferred embodiments, it is understood that variations and modifications will occur to those skilled in the art. Therefore, it is intended that the appended claims cover all such equivalent variations that come within the scope of the invention as claimed.

What is claimed is:

1. A method of treating a condition in a host that is responsive to an agonist, the method comprising administering a multi-layer pharmaceutical composition comprising an agonist and an antagonist thereof that are not in direct contact with one another in the intact form of the composition, wherein administration of the intact form of the composition to the host effectively treats the condition in a manner more efficacious than placebo when measure using the Brief Pain Inventory.

2. The method of claim 1 wherein the host is treated for up to twelve weeks.

3. A method of treating a condition in a host that is responsive to an agonist, the method comprising administering a multi-layer pharmaceutical composition comprising an agonist and an antagonist thereof that are not in direct contact with one another in the intact form of the composition, wherein administration of the intact form of the composition to the host effectively treats the condition in a manner more efficacious than placebo when measured using the WOMAC Osteoarthritis Index.

4. The method of claim 4 wherein the host is treated for up to twelve weeks.
